(19) United States
(12) Patent Application Publication
(10) Pub. No.: US 2008/0227128 A1
(43) Pub. Date: Sep. 18, 2008

(54) Resonance Energy Transfer System and Method

(75) Inventor: Dan P. Felsenfeld, New York, NY (US)

Correspondence Address: DARBY & DARBY P.C., P.O. BOX 770, Church Street Station, New York, NY, 10008-0770, US

(73) Assignee: Mount Sinai School of Medicine of New York University, New york, NY (US)

(21) Appl. No.: 11/576,375

(22) PCT Filed: Sep. 30, 2005
(86) PCT No.: PCT/US05/34925

§ 371 Date: Jan. 11, 2008

Related U.S. Application Data
(60) Provisional application No. 60/615,339, filed on Oct. 1, 2004.
(60) Provisional application No. 60/658,437, filed on Mar. 3, 2005.

(51) Int. Cl. C12Q 1/66; G01N 21/64

(52) U.S. Cl. 435/8; 436/172

(57) ABSTRACT

The present invention relates to a novel BRET system. The BRET system can be used to identify modified recognition sites within a protein insert and to identify the compounds involved in modulation of a given modification. The BRET system of the present invention can also be used in a screening method construct insert to identify candidates for drug development.

### CROSS-REFERENCE TO RELATED APPLICATIONS

Priority is claimed under 35 U.S.C. §119(e) to co-pending U.S. Provisional Patent Application Ser. No. 60/615,339, filed on Oct. 1, 2004 and 60/658,437, filed on Mar. 3, 2005. The contents of these priority applications are hereby incorporated into the present disclosure by reference and in their entirety.

### STATEMENT REGARDING FEDERALLY SPONSORED RESEARCH

The research leading to this invention was supported by the National Institute of General Medical Sciences of the National Institutes of Health, Grant No. GM63192. Accordingly, the United States government may have certain rights to this invention.

### FIELD OF THE INVENTION

The present invention relates to reporter systems and methods for their use based on resonance energy transfer. These systems employ a donor and an acceptor, which may be directly linked. The invention also encompasses use of such systems to detect an event, such as modification through molecular interaction, conformational change, or chemical modification, that is associated with an insert, such as a polynucleotide or polypeptide insert. The system is useful inter alia for screening to identify drug candidates and for studying cellular signaling pathways.

### BACKGROUND

### Fluorescence, FRET, and BRET

The physical phenomenon of fluorescence is the radiative decay process of a molecule in an electronically excited state. Depending upon the electronic structure of the particular molecule, when exposed to electromagnetic radiation (photons) of a first wavelength, the molecule may emit photons at a longer wavelength. The first wavelength is termed the excitation (or absorption) wavelength and is better thought of as a distribution of wavelengths (a spectrum) where the electronic excitation of the molecule overall increases to a maximum and then decreases as the wavelength of electromagnetic radiation approaches and then passes the optimal excitation wavelength. The longer wavelength of the emitted photon is also a spectrum, an emission spectrum, that is shifted to longer wavelengths compared to the absorption spectrum.

The process of fluorescence can begin with a molecule absorbing a photon. The energy absorbed places the molecule in a higher electronic energy state. Once in the higher electronic state, some of the absorbed energy is released through non-radiative decay, loss of energy due to thermal motion of the molecule. The rest of the energy may be lost through the spontaneous release of a photon. Since some energy of the original, absorbed photon was lost as thermal motion (heat), the released photon is of a lower energy, and thus the emission wavelength is a longer wavelength, than the original photon. When the release of a photon is immediate after the original photon was absorbed, the process is termed fluorescence. When the release occurs over a longer period of time, the process is termed phosphorescence and is due to the existence of a special electronic excited state not present in molecules that undergo fluorescence.

In fluorescence resonance energy transfer (FRET, also known as Forster resonance energy transfer), overlapping emission and absorption spectra of two molecules are utilized. A fluorescence donor molecule absorbs photons according to its absorption spectrum. The energy is partially released as thermal motion, and the remaining energy may be released as photons according to its emission spectrum. However, if there is a second fluorescence molecule in close proximity with an absorption spectrum that overlaps the emission spectrum of the first molecule, the first molecule (the donor) may transfer the energy, without the emission of a photon, to the second molecule (the acceptor). The greater the overlap of the spectra and the closer the proximity of the donor and acceptor, the more probable the energy transfer. The acceptor, having absorbed the transferred energy, loses some through thermal motion and the rest through emission of photons according to its emission spectrum. Therefore, if the donor and acceptor are not in close spatial proximity, the emission spectrum of the donor will dominate any observed signal in FRET. Conversely, should the donor and acceptor be close enough to transfer energy, the acceptor emission spectrum will dominate due to donor-acceptor interaction. The spatial limit of FRET is approximately 100 Å.

In bioluminescence resonance energy transfer (BRET), the concepts are the same as in FRET, except that the energy supplied through the donor to the acceptor (or released as a photon should they not be in close proximity) does not originate through the absorption of photons. The emission spectrum of the donor overlaps the absorption spectrum of the acceptor; however, the donor transfer energy is supplied through a chemical reaction. Fluorescence due to a chemical reaction is termed chemiluminescence; and when due to a chemical reaction involving biomolecular species (either as reactants or as catalysts), it is termed bioluminescence.

### FRET and BRET as Reporter Systems

FRET and BRET are techniques that do not destroy the sample to be tested but that report on molecular events, such as binding, chemical modification, and conformational change. The donor will transfer energy to the acceptor only if the donor is close enough to the acceptor to do so. Given a particular donor-acceptor pair, if a transfer of energy does occur, the donor and acceptor must have been close enough to interact, where a higher efficiency of energy transfer indicates a closer relationship between the donor and acceptor. Therefore, the event must have occurred for the donor and acceptor to be brought close enough together for the acceptor to fluoresce.

### Use of FRET as a Reporter System

FRET has been used extensively in reporter systems. As examples, FRET reporters that detect changes in free Ca²⁺ concentration (Ting et al. (2001) Proc. Natl. Acad. Sci. USA 98:15003-15008) and examine tyrosine kinase phosphorylation sites (Miyawaki et al. (1997) Nature 388:882-887) have been generated based on a donor-insert-acceptor design.

### Use of BRET as a Reporter System

BRET systems have been used as reporter systems in living cells (in vivo) and in the absence of living cells (in vitro). Xu et al. ((1999) Proc. Natl. Acad. Sci. USA 96:151-156), Angers et al. ((2000) Proc. Natl. Acad. Sci. USA 97:3684-3689) and Boute et al. (2001) Mol. Pharmacol. 60:640-645) used BRET to investigate protein-protein interactions in vivo and in vitro. Their systems used a Renilla luciferase (Rluc) genetically fused to a first protein of interest and a separate green fluorescent protein (GFP) mutant genetically fused to a second protein of interest (which may be identical to the first protein of interest or not). The Rluc enzyme catalyzes the oxidative decarboxylation of a coelenterazine, the substrate of Rluc, to produce a coelenteramide and photons and is thus the donor species.

The fusion proteins were produced through the design of DNA vectors to transcribe the two fusion proteins each as separate proteins. Using this system, the interactions of the proteins of interest (circadian clock proteins for Xu et al, β₂-adrenergic receptor for Angers et al., and the insulin receptor for Boute et al.) were studied. If the proteins of interest interacted, they would provide the requisite proximity between donor and acceptor and the efficiency of energy transfer would increase. Then, the acceptor emission would increase relative to the emission due to the donor, and the strength of the resulting fluorescence would be detected.

Single component BRET systems have been described in WO 01/46691, WO 01/46694, and WO 99/66324. However, there is a need for tyrosine and serine/threonine reporters of the present invention.

### FRET and BRET Reporter Systems

Some disadvantages in using FRET are spectrum-based. FRET systems are limited by the requirement for no significant overlap of the donor absorption spectrum with the acceptor absorption spectrum. Otherwise, excitation of the donor will simultaneously excite the acceptor, and the acceptor might emit a photon without energy transfer from the donor, increasing the likelihood of producing false positives.

Both FRET and BRET systems are also limited by the requirement for significant overlap of the donor emission spectrum with the acceptor absorption spectrum. If this emission-absorption overlap is insignificant, the efficiency of energy transfer would be low; and no photon would be emitted by the acceptor, even when the donor and acceptor are close in proximity. Thus, this situation would increase the likelihood of producing false negatives. Ideally, the emission spectrum of the donor should overlap as much as possible with the absorption spectrum of the acceptor.

Another spectrum-based limitation for both BRET and FRET systems is that the emission spectra of the donor and acceptor should not themselves overlap significantly. Such an emission-emission overlap would blur the distinction between a signal generated from the donor versus one generated by the acceptor. Photons released by the donor would cause a detectable signal (which would be a false positive) with the same emission spectrum as photons released by the acceptor (which would be a true positive signal).

Finally, the donor fluorophore in FRET may experience photobleaching upon excessive excitation. This bleaching would destroy the FRET system as a reporter molecule since the donor would not respond to its stimulus, namely photons.

Two-component systems require a donor attached to a molecule of interest and an acceptor attached to a second molecule of interest. Therefore, the proximity between donor and acceptor is provided by interaction, if any, of the two molecules of interest; and it is that interaction that is being tested. Often, however, a first molecule of interest is known; but molecules that interact with it are not. Therefore, if only one molecule is available, and it is unknown what other molecules interact with it, or even what the interaction itself is, this two-component system cannot then be used.

Therefore, there is a need for a system and method that does not have the absorption-absorption spectral disadvantage or photobleaching of FRET systems or the limitations of two-component systems. Further, there is a need for systems and methods that can be used to study events associated with a molecule that include, but are not limited to, binding; conformational change; phosphorylation, ubiquitination, acetylation, other post-translational modifications of proteins; and other chemical modifications. These events may also include, but are not limited to, cellular localization and systemic localization. There is a need for tyrosine kinase and threonine kinase reporters and reporters of other cell signaling events.

### SUMMARY OF THE INVENTION

The system of the present invention provides several advantages over current systems. If BRET is used, the FRET absorption-absorption overlap and photobleaching problems are not present, since the donor is excited through a chemical reaction, not electromagnetic radiation. Therefore, as a non-limiting example, excitation of the donor will not simultaneously excite the acceptor, avoiding false positives. Additionally, BRET reduces the detectable signal background due to, e.g., autofluorescence of the donor, since there is only one fluorescent species that can be excited by photons: the acceptor (or, more generally, there is only one species that displays a detectable signal, positive or negative).

Also, in the system of the present invention, the donor and acceptor molecules are conjugated to form a single composite molecule. The donor and acceptor can be attached directly to one another or may additionally comprise an insert between them. Events associated with the insert (when present) that can change the proximity of the donor to the acceptor may be, but are not limited to, binding; conformational change; phosphorylation, ubiquitination, acetylation, methylation, other post-translational modifications of proteins; and other chemical modifications. These events may also include, but are not limited to, cellular localization and systemic localization. As a further non-limiting example, a polypeptide segment may be used as the insert. This segment may undergo cellular phosphorylation.

The system of the present invention would allow the reporting of phosphorylation of the segment insert without direct knowledge of the pathway or cellular constituents involved. This, then, would facilitate identification of the pathway and constituents involved in the phosphorylation. For example, a particular enzyme that acts on a known or putative substrate may not be known. The system and method of the invention may be used to identify the enzyme.

As another non-limiting example, a polypeptide may be known to become phosphorylated, or the recognition sequence required for phosphorylation, may be unknown. The system of the present invention could be used to identify these. An application of the invention that is contemplated, but does not limit the invention, is that several inserts could be designed such that several recognition sites may be individually incorporated into BRET systems so that in separate trials the exact recognition sequence could be determined. These inserts can be quickly incorporated into composite molecules permitting a number of tests to be conducted in parallel. Thus, the present invention contemplates high-throughput methods, including the development of a library of reporter systems that can be used in arrays. The design of the insert of the present invention makes it possible to easily generate multiple reporter systems. Non-limiting examples of phosphorylation may be due to, but not limited to, tyrosine kinases or serine/threonine kinase pathways.

In one embodiment of the present invention, the reporter system is a BRET system comprising a donor-insert-acceptor resonance energy transfer system comprising a donor molecule, an insert molecule of interest attached to the donor, and an acceptor molecule attached to the insert species, wherein the donor molecule emits energy in the presence of a donor activator and the acceptor molecule displays a detectable signal in response to the emission of energy by the donor molecule and the co-occurrence of an event associated with the insert molecule, said event indicative of a property of the insert molecule.

Another embodiment of the present invention further comprises, in addition to the foregoing donor-insert-acceptor BRET system, a donor-acceptor BRET system comprising a donor molecule, and an acceptor molecule directly attached to the donor, wherein the second donor molecule emits energy in the presence of a second donor activator and the second acceptor molecule displays a detectable signal in response to the emission of energy by the donor molecule. This insert-free donor-acceptor system is meant for use as a control in conjunction with a donor-insert-acceptor system.

In a more specific embodiment the invention provides a donor-insert-acceptor resonance energy transfer system comprising:
(i) a donor molecule;
(ii) an insert molecule attached to said donor molecule, said insert molecule being a fragment of an actual or putative substrate of a tyrosine kinase or a serine/threonine kinase or a variant of said substrate; and
(iii) an acceptor molecule attached to said insert molecule;
   wherein said insert molecule maintains a predetermined spacing, r, between said donor molecule and said acceptor molecule within the range of 0.7R₀ to 1.1R₀, and wherein said donor molecule emits energy in the presence of a donor activator and said acceptor molecule displays a detectable signal in response to the emission of energy by said donor molecule and upon the co-occurrence of an event modifying the insert molecule to alter said spacing, r, resulting in a measurable change in said detectable signal, wherein said change is indicative of said event.

Another embodiment of the present invention is a donor-insert-acceptor system comprising a nucleic acid molecule encoding a donor polypeptide with an insert polypeptide attached to the donor polypeptide, and an acceptor polypeptide attached to the insert polypeptide of interest, wherein upon expression of the nucleic acid construct the donor polypeptide emits energy in the presence of a donor activator and the acceptor polypeptide displays a detectable signal in response to the emission of energy by the donor polypeptide and the co-occurrence of an event associated with the insert polypeptide, said event indicative of a property of the insert polypeptide.

In more specific embodiments, the system donor, acceptor, and insert, if present, can be proteins or DNA encoding proteins. Contemplated acceptor and donor proteins include, without limitation, autofluorescent proteins and luciferases, for example, but not limited to, green fluorescent protein and Renilla luciferase, respectively. In another specific embodiment, the insert of the BRET system comprises the amino acid sequence of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

Also provided by the present invention is a method of detecting an event associated with an insert in a donor-insert-acceptor resonance energy transfer system comprising the steps introducing a first donor-insert-acceptor system into a first test sample and a second system into a second control sample; introducing a donor activator into the first and second samples; measuring the detectable signal of the acceptor of the first system within the first test sample and of the acceptor of the second system within the second control sample; and determining a signal ratio for the first and second samples; wherein a difference in the signal ratio indicates that an event associated with the insert molecule has occurred.

In a more specific embodiment, the present invention provides a method of detecting an event associated with an insert in a donor-insert-acceptor resonance energy transfer system comprising the steps:
(i) measuring a first detectable signal of an acceptor of a first donor-insert-acceptor system according to claim 1 within a first test sample and a second detectable signal of an acceptor of a second donor-insert-acceptor system according to claim 1 within a second control sample; and
(ii) determining a signal ratio for the first and second samples; wherein a difference in the signal ratios indicates that an event associated with the insert has occurred.

Further provided by the present invention is a method of analyzing the signal ratio. This includes measuring the signal ratio within a sample that contains an additional molecule (the treated sample) and measuring the ratio within a sample that does not have the additional molecule (the untreated, or control, sample). This ratio of ratios is used in the following formula to determine the percent change compared to baseline:

$$100 \cdot ( \frac{treated sample ratio}{untreated sample ratio} - 1 ) .$$

In a further embodiment, the first and second samples of the methods of the present invention are cells, cell lysates, or cell-free preparations. For example, cell samples contain living cells; cell lysate samples contain cellular components but no intact cells; and cell-free preparations do not contain cells or cellular components but only molecules, which may or may not be isolated.

### BRIEF DESCRIPTION OF THE DRAWINGS

FIG. 1A is a schematic diagram of chimeric BRET constructs.

FIG. 1B is a bar graph that shows epidermal growth factor (EGF) significantly reduces the BRET ratio of the L1-BRET construct transfected in HEK-293 cells.

FIG. 1C is a bar graph that shows mutation of a tyrosine to one of aspartate, histidine, or phenylalanine residue abolishes the decrease in BRET ratio following stimulation of HEK-293 cells with EGF.

FIG. 1D is a graph that shows EGF reduces the BRET ratio of the L1-BRET construct in a dose-dependent manner.

FIG. 1E is a graph that shows EGF reduces the BRET ratio of the L1-BRET construct in a time-dependent manner.

FIG. 1F is a graph that shows the tyrosine kinase inhibitor, genistein, reverses the decrease in BRET ratio (i.e., results in a higher BRET ratio than that observed in the absence of genistein) following EGF stimulation of HEK-293 cells transfected with the L1-BRET construct. The dotted line represents the basal level (background) of BRET.

FIG. 1G shows immunoblots of the BRET constructs. HEK293 cells stably transfected with the L1-BRET or CHIM BRET constructs had genistein introduced (+) or not (−). Phosphorylated BRET constructs are detected in the upper blot. The expression levels of the two constructs are shown in the lower blot.

FIG. 1H is a graph illustrating the inverse relationship between the BRET ratio of the L1-BRET construct and the phosphorylation state of QFNEDGSFIGQY (“FIGQY”, SEQ ID NO: 1).

FIG. 2A is a graph that shows the MEK inhibitor PD98059 increases the BRET ratio of the L1-BRET construct transfected in HEK-293 cells in a dose-dependent manner.

FIG. 2B is a graph that shows the MEK inhibitor U0126 increases the BRET ratio of the L1-BRET construct transfected in HEK-293 cells in a dose-dependent manner.

FIG. 2C is a bar graph that shows mutation of the QFNEDGSFIGQY (“FIGQY”, SEQ ID NO: 1) tyrosine to an aspartate, QFNEDGSFIGQD (“FIGQD”, SEQ ID NO: 3); histidine, QFNEDGSFIGQH (“FIGQH”, SEQ ID NO: 4); or phenylalanine, QFNEDGSFIGQF (“FIGQF”, SEQ ID NO: 5) residue abolishes the increase in BRET ratio following inhibition of MEK with PD98059.

FIG. 2D is a graph that shows the MEK inhibitor PD98059 increases the BRET ratio of the L1-BRET construct transfected in ND7 cells in a dose-dependent manner.

FIG. 2E is a graph that shows the MEK inhibitor PD98059 increases the BRET ratio of the L1-BRET construct transfected in PC12 cells in a dose-dependent manner.

FIG. 2F is an immunoblot that shows tyrosine phosphorylation of endogenously expressed L1-CAM is dependent on the MAPK signaling pathway. Phosphorylated BRET constructs are detected in the upper blot. The same blot was stripped and re-probed with an Ab directed against L1-CAM for the lower blot.

FIG. 3A are images that show that treatment of transfected HEK-293 cells with EGF alone leads to a decrease in the level of ankyrin B recruited to the plasma membrane, and the decrease in the level of ankyrin B recruitment to the plasma membrane after EGF stimulation was reversed following the addition of PD98059 in the EGF containing preparations.

FIG. 3B is a bar graph that shows direct quantification of ankyrin B colocalization with L1-CAM at the cell membrane.

FIG. 3C is a bar graph that shows MAP kinase activity regulates L1-CAM-mediated neuronal growth in an ankyrin-dependent manner. However, growth on Ng-CAM was partially rescued by the addition of a peptide that inhibits L1-CAM-ankyrin interactions (AP-YF) while growth on laminin was unaffected by similar treatment.

FIG. 4A is a schematic diagram illustrating a hypothesis for the role of L1-CAM in neuronal growth.

FIG. 4B is a schematic diagram that shows hypothesis for the role of L1-CAM in neuronal growth.

FIG. 5A is a graph that shows the effect of the tyrosine kinase inhibitor genistein in reversing the decrease in BRET ratio of HEK-293 cells transfected with the L1-BRET construct.

FIG. 5B is a graph that shows the MEK (mitogen-activated protein kinase) kinase inhibitor PD98059 reverses the decrease in BRET ratio of HEK-293 cells transfected with the L1-BRET construct.

FIG. 5C is a bar graph that shows the MEK substrate (MEKSBS, which is [Biotin]—A-D-P-D-H-D-H-T-G-F-L-T-E-Y-V-A-T-R-W-[OH], SED ID NO: 14) and KPLGSDDSLADY peptide (SEQ ID NO: 6) are both phosphorylated in the presence of purified MEK in an in vitro kinase assay. The increase in phosphorylation is inhibited by the MEK inhibitor, U0126.

FIG. 6A is a graph that shows the erbstatin analog increases the BRET ratio of the L1-BRET construct in a dose-dependent manner.

FIG. 6B is a bar graph that shows the phosphotyrosine phosphatase inhibitor PAO decreases the BRET ratio of the L1-BRET construct.

FIG. 6C is a graph that shows the src-family tyrosine kinases inhibitor PP1 has no effect on the BRET ratio of the L1-BRET construct.

FIG. 6D is a graph that shows the src-family tyrosine kinases inhibitor PPs has no effect on the BRET ratio of the L1-BRET construct.

FIG. 7A is a bar graph that shows application of EGF significantly reduces the BRET ratio of an L1-BRET construct containing 25 residues of the L1 cytoplasmic sequence and a myristoylation site located upstream of the GFP coding region.

FIG. 7B is a bar graph that shows mutation of the DDSLADYGGSVDVQFNEDGSFIGQY (“myr-FIGQY”, SEQ ID NO: 7) tyrosine to a histidine, DDSLADYGGSVDVQFNEDGSFIGQH (“myr-FIGQH”, SEQ ID NO: 8) or phenylalanine, DDSLADYGGSVDVQFNEDGSFIGQF (“myr-FIGQF”, SEQ ID NO: 9) residue abolishes the increase in BRET ratio of the myristoylated construct following inhibition of MEK with U0126.

FIG. 7C is a schematic diagram of the myristoylated chimeric BRET constructs with inserts.

FIG. 8 is a bar graph which shows the application of EGF significantly reduces the BRET ratio of HEK-293 cells transfected with the SACT-A construct (insert of QFNEDGSFIGQY, SEQ ID NO: 1) but not with the SACT-B (insert of NEDGSFIGQYSG, SEQ ID NO: 10) or SACT-C (insert of DGSFIGQYSGKK, SEQ ID NO: 11) constructs.

FIG. 9A is a table that shows the application of EGF significantly reduces the BRET ratio of the KGGKY construct transiently transfected in HEK-293 cells. The tyrosine kinase inhibitor genistein significantly increases the BRET ratio of the KGGKY construct. The MEK inhibitor PD-98059 has no effect on the BRET ratio of the KGGKY construct.

FIG. 9B is a graph that shows the src-family tyrosine kinase inhibitor, PP1, but not PP2, increases the BRET ratio of the KGGKY construct in a dose-dependent manner.

FIG. 9C is a bar graph that shows the response of the BRET reporter depends on Src expression. Fibroblasts derived from wild-type (+/+src) or Src-null (−/−src) mice were treated with FGF.

FIG. 10A is a graph that shows the PKA inhibitor H-89 increases the BRET ratio of the PKA construct transfected in HEK-293 cells in a dose-dependent manner. Mutation of the terminal serine residue to an alanine abolishes the increase in BRET ratio following treatment of HEK-293 cells with H-89.

FIG. 10B is a graph that shows the myristoylated PKA inhibitor peptide (myrPKAI) increases the BRET ratio of the PKA construct in a dose-dependent manner. Mutation of the serine residue to an alanine abolishes the increase in BRET ratio following treatment with myrPKAI.

FIG. 10C is a graph that shows the src-family tyrosine kinase inhibitor PP1 has no effect on the BRET ratio of the PKA constructs.

FIG. 10D is a bar graph that shows the PKA activator Sp-cAMPS (Adenosine-3′,5′-cyclic monophosphorothioate, Sp− isomer), increases the BRET ratio of the PKA construct, but has no effect on the PKA-Ala construct.

FIG. 11. Plot showing the required increase in separation of the donor and acceptor from a starting point (r/R0) required to give a change in FRET of a given percentage (indicated by dashed curves). Bold curve indicates the FRET efficiency at a given distance (r/R₀; left X axis). Dashed curves indicate the increase in separation (r/R0; right X axis) needed to produce a change in E of the percentage indicated for each curve. Therefore, starting at a separation of 0.86 r/R0, one would need an increase in distance of 0.17 r/R0 to produce a 25% change in signal. For an R0 of 50 Å (an accepted value for GFP-related fluorophores), an increase in 8.3 Å would yield a 25% decrease in signal.

To put this simply, if the donor and acceptor start at a separation near R0, a very small change in separation yields a large change in FRET signal. This is due to the large non-linearity (E varies in inverse proportion to r⁶) of the curve around R₀. All calculations are based on the Forster equation:

$$E = \frac{{R}_{0}^{6}}{{R}_{0}^{6} + {r}^{6}} ( Lakowicz , 1999 ) .$$

### DETAILED DESCRIPTION

The present invention relates to a resonance energy transfer system and method for its use in detecting events associated with one (or more) molecules of interest. For example, the invention encompasses use of such systems to detect an event, such as modification through molecular interaction, conformational change, or chemical modification, that is associated with an insert, such as a polynucleotide or polypeptide insert. The system is useful for screening to identify drug candidates, for identifying molecules that interact with others and for studying cellular pathways. The system of the present invention may be used to detect modification of a polypeptide of interest, its participation in a pathway, or may be used to screen for drug compounds. The present invention may be used within an animal, a living cell or tissue, or in the absence of living cells using fully or partially purified molecules.

### DEFINITIONS

A “resonance energy transfer system” of the present invention comprises an energy (e.g., fluorescence) donor molecule and an energy acceptor molecule, which upon acceptance of a sufficient amount of energy displays a detectable signal. The donor and acceptor may be attached to one another directly (a “donor-acceptor resonance energy transfer system”) for a control system or the donor may be attached to an insert; and the insert may then be attached to the acceptor (a “donor-insert-acceptor resonance energy transfer system”). The donor releases energy in the presence of a donor activator and the acceptor displays a detectable signal in response to (a) the energy emission by the donor species and (b) the co-occurrence of an event associated with the insert such as a modification to the insert, which is the subject of the inquiry. The system of the present invention may comprise without limitation protein molecules. The system of the present invention may also comprise nucleic acids such as DNA that encodes protein molecules. In another embodiment, the system of the present invention may comprise any molecule that acts as an energy donor attached to any molecule that acts as an energy acceptor and may include any molecule that is attached to the donor and acceptor molecule as an insert, i.e., a molecule of interest. The donor and acceptor may be BRET or FRET donor-acceptor pairs, including, but not limited to, Rluc-GFP2 and CFP-YFP, respectively.

A “donor” is a molecule that is capable of transfer of energy to another molecule, for example through resonance energy transfer. The energy may be initially absorbed as a photon or may be energy released by the donor due to a chemical reaction. A non-limiting example of a donor would be a fluorescence donor molecule that can either transfer the energy by resonance energy transfer or by emitting a lower energy photon.

An “acceptor” is a molecule that is capable of accepting energy transferred from a donor molecule and emitting, e.g., a photon of lower energy or displaying another detectable signal. A non-limiting example of an acceptor would be a fluorescence acceptor molecule, capable of absorbing a photon of higher energy and emitting a photon of lower energy (the standard fluorescence process) in the absence of a donor.

An “insert” is any molecule that is attached between a donor and an acceptor. Non-limiting examples include a polypeptide or a nucleic acid molecule that undergoes a modification that changes the signal ratio. Another example is a molecule with a carbon-carbon double bond that changes from the trans configuration to the cis configuration wherein the isomerization changes the signal ratio.

A “donor activator” undergoes or initiates a process, for example, a chemical reaction, that results in the emission by the donor of a photon or transfer of energy through resonance energy transfer, or other method. A non-limiting example of a donor activator is a coelenterazine molecule, the substrate for Renilla luciferase. Another non-limiting example of a donor activator is a photon, as is the case for a FRET donor.

An “autofluorescent protein” is a protein that is capable itself of fluorescence. Non-limiting examples of autofluorescent proteins include green fluorescent protein (GFP) and mutants of GFP, such as GFP2. Autofluorescent proteins are translated from RNA and fold into their three-dimensional structures. No separate chemical entity is appended to the protein; rather, amino acid side chains of the protein react to form fluorescent moieties (fluorophores). Therefore, only the amino acid sequence is required.

A “system” comprises at least a donor and an acceptor, and can additionally include an insert. In a “BRET system,” the donor can transfer energy due to bioluminescence.

A “detectable signal” is a signal that is associated with the acceptor and donor molecules (and the emission of energy by the donor and receipt of energy by the acceptor). A ratio of the detectable signal of the acceptor over the detectable signal of the donor measures the efficiency of energy transfer, using, e.g., fluorescence. Another non-limiting example includes a first acceptor fluorescence signal divided by a first donor fluorescence signal divided again by a second acceptor fluorescence signal which was divided by a second donor fluorescence signal. This latter ratio, which is a ratio of ratios, may be used, as a non-limiting example, in an experiment where one BRET system contains an insert of interest whereas another BRET system is used as a control. Another non-limiting example includes using the formula

$$100 \cdot ( \frac{treated sample BRET ratio}{untreated sample BRET ratio} - 1 )$$

to determine the percent change compared to baseline in a BRET system. In each case, the ratios may be used to divide out background, or baseline, interferences. A measurable change in a ratio of detectable signals indicates that an event associated with the insert has occurred. Preferably the change is a statistically-significant change as illustrated in the working Examples below.

“Detecting the acceptor detectable signal” is by any method used to observe or measure acceptor detectable signal. Examples include, but are not limited to, using fluorescence spectrometers or “electromechanical plate readers” where the detectable signal is fluorescence. An electromechanical plate reader, such as the PerkinElmer Fusion™ Universal Microplate Analyzer, can observe and measure many samples simultaneously.

“Event associated with an insert” refers to any change in, modification of, or event involving an insert that is detectable by the system of the present invention and that correlates with the question asked by the method of the present invention. The changes may be, for example, but not limited to, a change in the conformation of the insert, a change in the electrical charge of the insert, cellular or systemic localization, binding of the insert, and/or a change in the chemical identity of the insert. A change in the chemical identity of the insert may be through post-translational modification of the insert (which may include, but is not limited to, acetylation, phosphorylation, ubiquitination, methylation, or glycosylation) if the insert is a protein sequence. Some processes or interactions may do several of these: Phosphorylation also changes the electronic charge of the insert, which could also alter the conformation of the insert. The event modifies the insert and thereby alters the spacing between donor and acceptor, which affects the detectable signal.

An “actual or putative substrate” is a substrate for an enzyme that is either known to be or suspected of being a substrate for that enzyme, respectively. Embodiments of the present invention include using the present invention to investigate unknown enzymes that act on known substrates or to investigate which substrates a known enzyme will act upon. Studies using the present invention can include investigations of particular pathways involved in certain interactions or events.

A “spacing, r” is the distance between the donor molecule and acceptor molecule. This distance may be given in units of length, such as Angstrom. The spacing, r, may also be given in terms of R₀, the distance at which the efficiency of energy transfer between a donor molecule and an acceptor molecule is 50%. This is roughly around 50 Å for Rluc and a GFP, but is dependent upon the particular donor-acceptor pair. Therefore, the spacing, r, may be given as a number multiplied by R₀.

Amino acids that are “replaced” may be, as non-limiting examples, mutated, shifted, or deleted. Amino acids may be chemically modified (incorporation of non-natural amino acids is a non-limiting example).

A “recognition site” is a region of a first molecule that is recognized by one or more other molecules. This recognition involves interaction between the first and at least one other molecule. The recognition site may be, for example, but not limited to, a binding site, a cleavage site, or a site for post-translational modification. The interaction may be, for example, but not limited to, binding, cleavage, phosphorylation, ubiquitination, methylation, or acetylation.

“Mitogen-activated protein kinase (MAPK) pathway” is a cellular pathway that has the general structure of stimulus→MAPK kinase kinase (MAPKKK)→MAPK kinase (MAPKK)→MAPK→biological response. The stimulus, which may be an activated G coupled protein receptor or other molecule, activates the MAPKKK to phosphorylate the MAPKK, which, now activated, in turn phosphorylates and activates the MAPK. Activated MAPK interacts with other molecules to produce a biological response, which can be, but is not limited to, cell differentiation, cell proliferation, cell movement, and cell death. The stimulus may be produced by cell receptors or other molecules. Therefore, the MAPK pathway can be sensitive to extracellular signals. Examples of MAPK pathways include the mitogen-activated protein kinase/ extracellular signal-regulated kinase (MAP/ERK) pathway, the stress-activated protein kinase/Jun N-terminal kinase (SAPK/JNK) pathway, and the p38 pathway. For the MAPK/ERK (MEK) MAPK pathway, the MAPKKK is Raf, the MAPKKs are MEK1 and MEK2, and the MAPKs are ERK1 and ERK2. For the SAPK/JNK pathway, the MAPKKKs, MAPKKs, and MAPKs are, respectively, MEK1, MEK4, MLK3, and AKS1→MKK4 and MKK7→SAPK/JNK1, SAPK/JNK2, SAPK/JNK3. Finally for the p38 MAPK pathway, these are MLK3, tousled-like Serine/threonine-protein kinase (TLK), DLK→MKK3 and MKK6→p38 MAPK.

A “MAPK pathway recognition site” is a recognition site that is recognized by a member of a mitogen-activated protein kinase (MAPK) pathway.

The “Src pathway” is a cellular pathway that involves a Src protein. Srcs are non-receptor tyrosine kinases, including, but not limited to, Fyn, Lck, and Yes. Srcs tend to be downstream of membrane-linked receptors and are involved in, for example, but not limited to, growth factor signaling.

A “Src pathway recognition site” is a recognition site that is recognized by a member of the Src pathway.

The donor, insert, or acceptor of the present invention may also act as epitope tags. A non-limiting example includes GFP2 as the acceptor. GFP2 may be used as an epitope tag wherein antibodies against GFP2 can be used to independently locate the BRET system.

A “sample” is any environment in which the BRET system of the present invention may be used. By way of non-limiting example, a sample may be or contain a tissue, a cell, a cell lysate, or a cell-free preparation containing a medium or a solvent (usually water) plus other molecules in the absence of cellular components or intact cells.

“Identical” in terms of molecular species means that the molecules are chemically the same. In terms of cells, “identical” means that the cells are of the same cell type or have the same genome.

“Screening compounds” means applying the methods of the present invention to determine if one or more particular compounds have a particular activity or other property.

A “nucleic acid molecule” (or alternatively “nucleic acid”) refers to the phosphate ester polymeric form of ribonucleosides (adenosine, guanosine, uridine, or cytidine: “RNA molecules”) or deoxyribonucleosides (deoxyadenosine, deoxyguanosine, deoxythymidine, or deoxycytidine: “DNA molecules”), or any phosphoester analogs thereof, such as phosphorothioates and thioesters, in either single-stranded form, or a double-stranded form. Oligonucleotides (having fewer than 100 nucleotide constituent units) or polynucleotides are included within the defined term as well as double-stranded DNA-DNA, DNA-RNA, and RNA-RNA segments. This term, for instance, includes double-stranded DNA found, inter alia, in linear (e.g., restriction fragments) or in circular DNA molecules (such as plasmids) and in chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

A “recombinant DNA molecule” is a DNA molecule that has undergone a molecular biological manipulation. The recombination may be natural (e.g., through naturally occurring recombinases) or man-made.

As used herein, the term “polypeptide” refers to an amino acid-based polymer, which can be encoded by a nucleic acid and prepared by expressing the nucleic acid or can be prepared synthetically. Polypeptides can be proteins, protein fragments, chimeric proteins, and amino acid-based polymers that do not correspond to a protein or protein fragment, etc.

The term “antibody”, or “Ab”, as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain comprises a heavy chain variable region (abbreviated herein as VH) and a heavy chain constant region. The heavy chain constant region comprises three domains, CH1, CH2 and CH3. Each light chain comprises a light chain variable region (abbreviated herein as VL) and a light chain constant region. The light chain constant region comprises one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

Antibodies may be polyclonal or monoclonal. Polyclonal antibodies recognize a particular molecule but recognize different binding regions, or “epitopes”, on the particular molecule. Monoclonal antibodies recognize the same epitope on a particular molecule.

### System Design

Design of constructs or systems of the present invention, which include the donor and acceptor molecules, is governed by several principles. For example, the minimum insert length is dependent upon the size of the recognition site of the molecule. A smaller insert encodes for a smaller recognition domain which could concomitantly decrease the selectivity or recognition of potential binding partners. The maximum insert length is the maximum distance at which efficient energy transfer between the FRET/BRET donor and FRET/BRET acceptor is possible, wherein the transferred energy is sufficient to cause display by the acceptor of the detectable signal. This distance is about 100 Å. For a protein insert, each residue in a fully extended polypeptide chain has a length of about 3.63 Å (T. Creighton (1984) Proteins: Structure and Function, W. H. Freeman: New York). Therefore, the maximum size insert, of a fully extended protein insert, is about 27 amino acid residues. Based on these principles, a peptide insert is preferably between about 10 and about 25 amino acids in length. Preferably, the encoded peptide insert, if up to 27 amino acids, should not have any secondary structure. Larger protein inserts are possible, if the protein (e.g., upon occurrence of the event associated with the insert) does assume higher order structure so that the donor and acceptor can still interact (see Ting et al. (2001) Proc. Natl. Acad. Sci. USA 98:15003-15008; Boute et al. (2001) Mol. Pharmacol. 60:640-645). However, as the sequence length increases, the exact recognition sequence may become more difficult to determine.

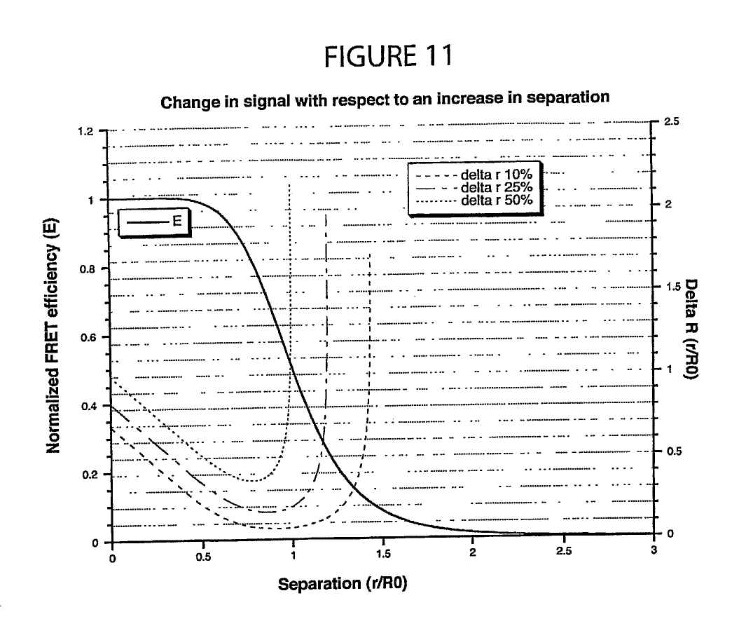

The above may be described in terms of FIG. 11. The sigmoidal curve is the efficiency of energy transfer plotted against the spacing, r, between a given donor molecule and a given acceptor molecule in terms of R₀. When E=1, the efficiency is 100%. R₀ is the r value at the position of the sigmoidal curve where the efficiency is equal to 50%. Therefore, r/R₀ at this position is equal to one. The other curves represent constant changes in signal and illustrate how, for different values of r/R₀, an incremental change in r may or may not be accompanied by a large change in signal. At very low and at very high efficiency values the change in r has to be quite large before a significant change in signal will occur, if indeed it occurs at all. But at r values relatively close to R₀ (such that r/R₀ is close to 1) a small change in r will bring about a large change in signal. Therefore, the insert should be preferably of such a size that it would maintain a spacing r between donor and acceptor close to R₀.

In more detail, the change to be expected in a FRET or BRET signal due to a modification of an insert of the present invention, a given donor-acceptor pair, can be illustrated in the three regions of the sigmoidal curve. In the region where E in near 1 (r/R₀ from 0 to about 0.3), the spacing between the donor and acceptor will always allow transfer of energy. Therefore, movement of the donor relative to the acceptor within this range will not give any change in signal; the acceptor signal will always be present. In the region where E is near 0 (r/R₀ from about 2.4 and beyond), there is no transfer of energy. The donor and acceptor may move great distances relative to one another; however, as long as r/R₀ is greater than about 2.4, energy transfer will not occur; and the acceptor will not be excited. Thus, the acceptor signal will always be absent. In the transition region of ˜0.3<r/R₀<˜2.4 (particularly between 0.7R₀ and 1.1R₀), a change in acceptor signal will be observed when the donor moves relative to the acceptor. The amount of signal change is indicated by the thin parabolic curves. Where the parabolic curves intersect the sigmoidal curve, this is the r/R₀ value at which the given percent change in signal will occur. Traveling from r/R₀=0 to r/R₀=1, the signal change increases to 50%. Continuing from r/R₀=1 to greater values of r/R₀, the change in signal decreases again. The initial increase followed by decrease in signal change is expected since in the plateau regions of the efficiency plot, no change occurs. It is within the transition region of the sigmoidal curve, particularly around r/R₀=1, then, where the greatest change in signal will be observed. The experimental direction of change in signal, then, can also be used to indicate where on the sigmoidal plot a particular system lies. (See Stryer et al. (1967) Proc. Natl. Acad. Sci. USA 58:719-726 incorporated by reference in its entirety; Lakowicz (1999) Principles of Fluorescence Spectroscopy. 2^{nd} ed. Plenum: N.Y.)

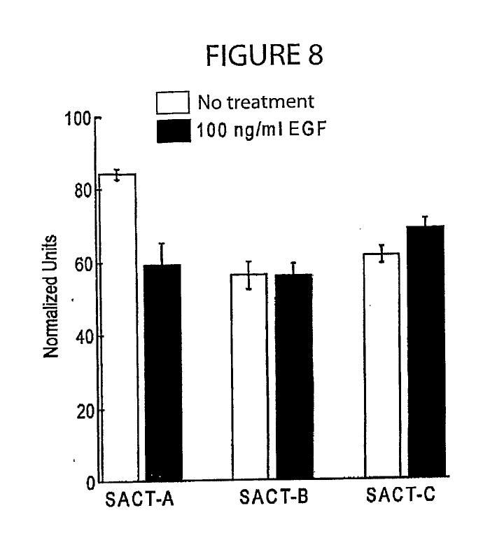

Finally, for polypeptide inserts modified by a chemical post-translational modification, the modified residue should be positioned as the C-terminal residue within the insert for there to be a change in the detectable signal from the acceptor. Using other amino acid positions was found to be less successful or unsuccessful in changing the acceptor detectable signal and thus the BRET ratio (see Example 6 and FIG. 8, where the results shown are mean±standard deviation, n=5).

Selection of the donor and acceptor depend upon the particular experimental set-up and are governed by the principles described within “FRET and BRET Reporter Systems,” above. Donor-acceptor pairs may be used where the donor is any bioluminescent or fluorescent moiety and the acceptor is any appropriate fluorophore acceptor. FRET donor-acceptor FRET pairs include, but are not limited to, fluorescein and rhodamine, ECFP (available from Clonetech) and YFP. Venus YFP is described in Nagai et al. (2002) Nat Biotechnol. 20:87-90 and Rekas et al. (2002) J Biol Chem. 277:50573-8, and its coding region is given in SEQ ID NO: 20. Donor-acceptor BRET pairs include Renilla luciferase and GFP mutants. SEQ ID NO: 17 gives the protein sequence of a FRET donor-acceptor system using the ECFP and Venus YFP described above. SEQ ID NO: 19 gives the nucleic acid sequence of a plasmid encoding this system. SEQ ID NO: 16 gives the same FRET donor-acceptor pair with an insert which includes the sequence FIGQY.

In accordance with the present invention, there may be employed conventional molecular biology, microbiology, recombinant DNA, immunology, cell biology and other related techniques within the skill of the art. See, e.g., Sambrook et al. (2001) Molecular Cloning: A Laboratory Manual. 3^{rd} ed. Cold Spring Harbor Laboratory Press: Cold Spring Harbor, N.Y.; Sambrook et al. (1989) Molecular Cloning: A Laboratory Manual. 2^{nd} ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; Enna et al. eds. (2005) Current Protocols in Pharmacology John Wiley and Sons, Inc.: Hoboken, N.J.; Hames et al. eds. (1999) Protein Expression: A Practical Approach. Oxford University Press: Oxford; Freshney (2000) Culture of Animal Cells: A Manual of Basic Technique. 4^{th} ed. Wiley-Liss; among others. The Current Protocols listed above are updated multiple times every year.

### Compound Screening

The system of the present invention can be employed in screening methods to identify useful compounds, such as new drug candidates. The design of the present insert permits the rapid generation of new reporter constructs and their use to evaluate compounds. The parental construct, as a non-limiting example, may consist of the BRET acceptor (GFP2) and donor (Renilla luciferase; Rluc) concatenated with two unique restriction sites located in the intervening sequence. This chimeric construct (CHIM, nucleic acid sequence set forth in SEQ ID NO: 2, amino acid sequence set forth in SEQ ID NO: 15), expressed in a pcDNA3.1 Hygro (+) expression vector (Invitrogen), can serve as a positive control for BRET experiments.

The present invention can be used in high throughput screening (HTS) to identify, for example, compounds that are active in modulating neuronal growth and are thus potential drug candidates or for use in treating disorders that are regulated by the MAP kinase pathway including corpus callosum hypoplasia, mental retardation, and spastic paraplegia. A non-limiting example is that a polypeptide sequence could be reverse transcribed. That is, a nucleic acid sequence that gives rise to the polypeptide could be devised. This nucleic acid sequence could also be easily randomized. The small size of the insert allows for use of synthesized oligos which are readily produced using current techniques known in the art. Also, as a non-limiting example, the unique restriction sites engineered into the BRET system described in the Examples below allow for directional insertion as close to the GFP2 and Rluc coding regions as possible.

The present invention can be used for the screening of compound libraries to identify potential candidates for drug development. The change in signal ratio when the system of the present invention is contacted with a compound may identify drugs or pharmaceutically active compounds and may identify their effect on a cellular pathway. Also, the present invention can be used for the determination of the dosage dependence of these drug compounds, as is detailed below in the Examples.

Electromechanical plate readers can be used to detect signal ratio changes. Such plate readers can be employed for high throughput screening, drug candidate screening, and drug dosage dependence studies using the system of the present invention. Examples of plate readers that can be used in practicing the present invention include the Fusion™ family of plate readers offered by PerkinElmer (Boston, Mass.), including the PerkinElmer Fusion™ Universal Microplate Analyzer devices. The PerkinElmer EnVision™ HTS model can also be employed in practicing the present invention.

Plate readers detect a change in emitted fluorescent light frequency and use this information to provide signal ratio information. The plate readers can accommodate multi-well plates with tens, hundreds, or more samples per plate. Micro array plates may have thousands of samples per plate. Each sample is individually or simultaneously irradiated with electromagnetic radiation at a frequency according to the absorption spectrum of the donor. One or more detectors are used singly or simultaneously, respectively, to detect the resulting fluorescence and determine the signal ratio. These plate readers can be used with cell based assays, solution based assays, enzyme assays, reporter gene assays, immunoassays, binding studies, or molecular biology assays and can easily be scaled-up for industrial applications.

### Use of the Present Invention with Post-translational Modifications or Cellular Pathways

The system of the present invention can also be used for the detection of post-translational modification events in cellular pathways, including the MAP kinase pathway as described below in Example 2. The recognition site defined by the insert may be designed based on any recognition site believed to be functionally important for a particular pathway and a particular post-translational modification. For example, the data of Examples 4 and 5 illustrate that the present invention could be used with the src pathway and for the detection of tyrosine phosphorylation by src-family kinases.

The present invention is further described in the following working examples. However, the use of this and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing the invention in spirit or in scope. The invention is therefore to be limited only by the terms of the appended claims along with the full scope of equivalents to which those claims are entitled.

### EXAMPLES

### Materials

Rabbit anti-GFP polyclonal Ab was obtained from Molecular Probes (Eugene, Oreg.). Rabbit anti-phosphotyrosine polyclonal Ab was obtained from Upstate Biotechnology, Inc. (Lake Placid, N.Y.). Rabbit anti-Ll polyclonal Ab was a gift from Carl Lagenaur (University of Pittsburgh, Pittsburgh, Pa.; Lagenaur et al. (1987) Proc. Natl. Acad. Sci. USA 84:7753-7757). Mouse anti-myc monoclonal Ab was obtained from Developmental Studies Hybridoma Bank (University of Iowa, Iowa City, Iowa). Horseradish peroxidase (HRP)-conjugated goat anti-rabbit Ab was obtained from Jackson InumunoResearch Laboratories (West Grove, Pa.). Donkey anti-mouse Ab conjugated to indocarbocyanine Cy3 and donkey anti-rabbit Ab conjugated to indodicarbocyanince Cy5 were obtained from Jackson ImmunoResearch Laboratories. Human embryonic kidney (HEK)-293 and rat pheochromocytoma (PC)12 cells were obtained from American Type Culture Collection (Manassas, Va.; ATCC accession Nos. CRL-1573 and CRL-1721, respectively). Genistein, PD98059, PP1, PP2, and U0126 were obtained from BioMol Research Laboratories Inc. (Plymouth Meeting, Pa.). Epidermal growth factor (EGF) and nerve growth factor (NGF) were obtained from Sigma-Aldrich (St. Louis, Mo.). The codon humanized pRIuc and GFP2 vectors were obtained from BioSignal Packard (now PerkinElmer Life Sciences (Boston, Mass.)).

### Example 1

### BRET Reporter System

### BRET Construct Design

BRET constructs were designed using vectors encoding Renilla luciferase (the BRET donor) and green fluorescent protein 2 (GFP2, the BRET acceptor). (The GFP2 was Sapphire GFP.) Coding regions from each individual vector were copied by polymerase chain reaction (PCR) with additional restriction sites, permitting their ligation into a single, concatenated coding region (GFP2:Rluc) between Nott and Xhol sites in a pcDNA3.1 Hygro (+) eukaryotic expression vector (Invitrogen, Carlsbad, Calif.).

The chimeric (parental and control) BRET construct was generated as follows: The coding sequence from codon humanized pGFP2-N1 (BioSignal Packard; now Perkin Elmer) was amplified using PCR using primers:

| | |
|---|---|
| GCCCGCGGCCGCAATGGTGAGCAAGGGAGAG | (UPPER) |
| and | |
| CGCCCTCGAGTCCGGACTTGTACAGCTCGTCCAT | (LOWER) |

The product of this PCR reaction was inserted into a pGEM11ZF (Promega) vector using Notl and Xho1 sites (pGEM-GFP2).

The Rluc coding from codon humanized pRluc-N1 (BioSignal Packard; now Perkin Elmer) was amplified using PCR using primers:

| | |
|---|---|
| TAATCCGGAGGCGCGVVAATGACCAGCAAGGTGTAC | (UPPER) |
| and | |
| GGCGGCCTCGAGTCTAGATCGAATTCTTACT | (LOWER) |

The product of this reaction was inserted into the pGEM-GFP2 construct using BspE1 and Xho1 sites. The GFP:Rluc coding reion in the resulting construct (pGEM-BRET-CHIM) was excised using Not1 and Xho 1 and ligated into pcDNA3.1 Hygro (+) (Invitrogen).

This chimeric donor-acceptor construct (CHIM, SEQ ID NO: 2, amino acid sequence set forth in SEQ ID NO: 15) encodes unique BsrGI and Ascl sites in the intervening sequence between the Rluc and GFP2. These unique restriction sites were specifically engineered to be as close to the GFP2 and Rluc coding regions as possible. The Asc1 restriction site encodes for an additional proline and tyrosine between the insert and Rluc.

New constructs were generated by digesting the CHIM construct (SEQ ID NO: 2) with the restriction enzymes BsrG1 and Asc1 that opens the construct at the junction between the GFP2 and Rluc coding regions. The coding regions for the insert/reporter domain were generated by synthesizing complementary oligonucleotides that encode the reporter protein sequence in question. To the upper oligonucleotide sequence, the bases GTACAAG were added at the 5′ end and GG at the 3′ end. To the lower oligonucleotide, the bases CGCGCC were added to the 5′ end and CTT at the 3′ end. These additions serve to generate 5′ sticky ends that hybridize directly with the complimentary restriction sites in the digested CHIM vector. The complimentary oligonucleotides were mixed in an equimolar ratio, heated to 94° C. in a PCR machine and permitted to cool in steps to room temperature (94° for 4 min; 74° for 4 min; 68° for 4 min; allow to cool to room temperature). The cooled oligonucleotide mixture was ligated into the digested CHIM vector using the Rapid DNA Ligation kit (Roche). As oligonucleotides are generally synthesized without a terminal phosphate, it is essential to omit the alkyline phosphatase (CIP) digestion of the CHIM vector, as the vector, not the insert, is providing the phosphate groups necessary to complete the ligation.

After a 30 minute ligation, ligation mixtures were transformed into competent bacteria, and harvested by mini-prep the following morning. Although success rate using this protocol is essentially 100%, the new constructs can be tested for the addition of the insert as follows. 2-3 μg of miniprep DNA was digested with Not1 and Asc1 releasing the GFP2 and reporter insert domains. As a negative control, the CHIM vector was cut in parallel. The digest products were run on a 2% agarose gel, ensuring that the dye front was run as close as possible to the bottom of the gel. This generally permits the observation of a small band shift in the size of the restriction product by direct comparison to the CHIM construct (despite the very small ca. 30 bp size of the oligonucleotide inserts).

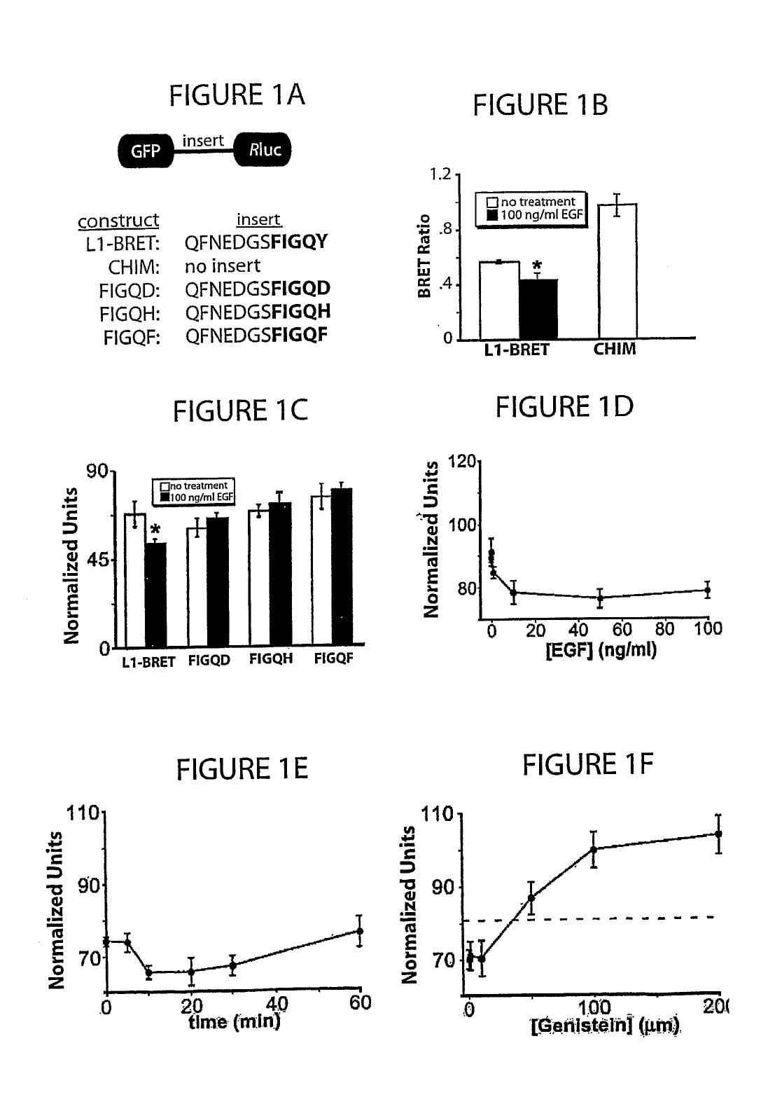

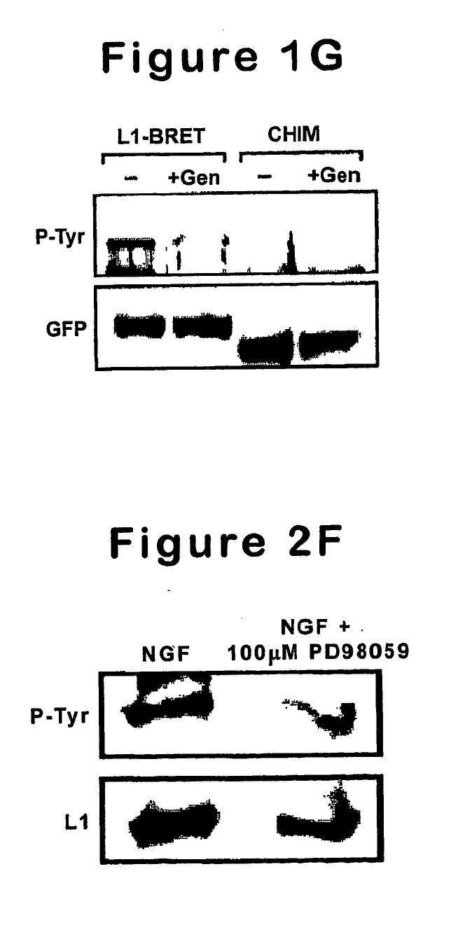

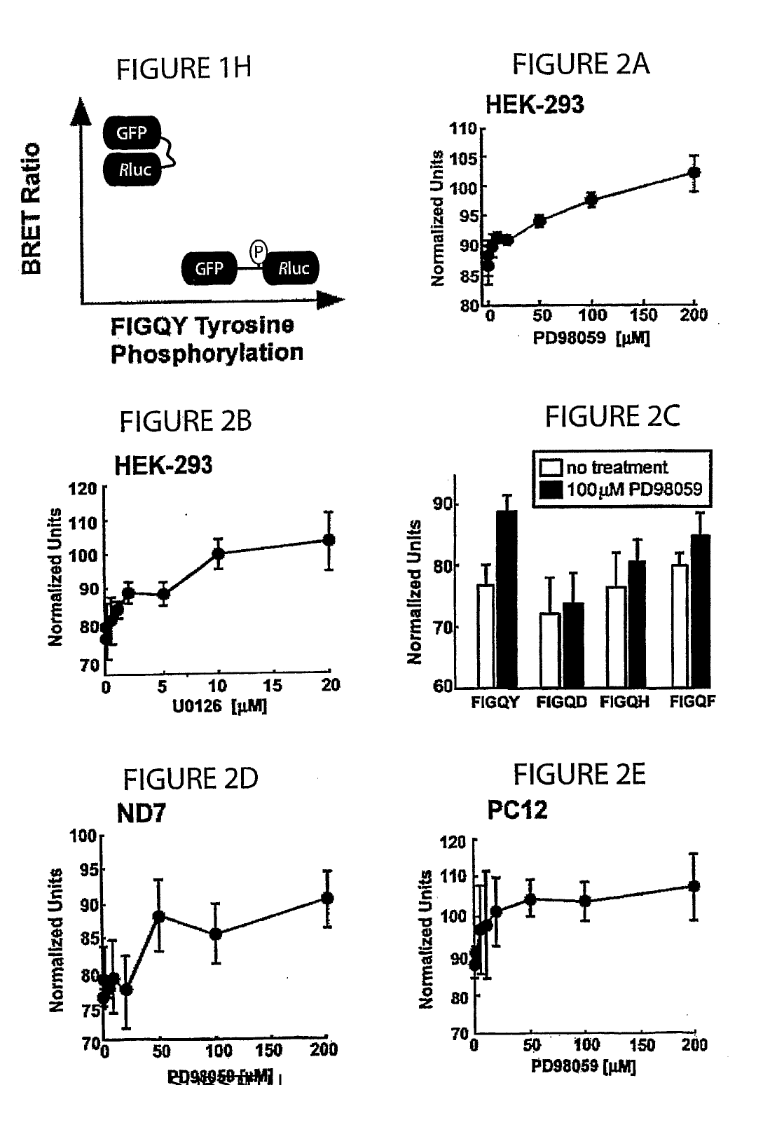

FIG. 1A shows various constructs generated. CHIM has no insert. SEQ ID NO: 2 represents the full GFP2-Rluc CHIM vector without an insert, where the amino acid sequence is set forth in SEQ ID NO: 15. The L1-BRET (L1-CAM BRET) construct has an insert of QFNEDGSFIGQY (SEQ ID NO: 1, plasmid sequence given in SEQ ID NO: 18) between the GFP2 and Rluc. Other inserts include QFNEDGSFIGQD (SEQ ID NO: 3), QFNEDGSFIGQH (SEQ ID NO: 4), and QFNEDGSFIGQF (SEQ ID NO: 5).

### BRET Assay

Near-confluent cultures of HEK-293 cells were harvested with trypsin-EDTA (0.05% trypsin, 0.53 mM EDTA; Invitrogen Corporation, Carlsbad, Calif.) and resuspended to a density of 2.5×10⁵ cells/ml. Aliquots (200 μl) of cell suspensions were added to white 96-well culture plates (CulturPlate™; PerkinElmer Life Sciences, Boston, Mass.) and incubated for 12 hrs at 37° C. HEK-293 cells were transfected with 0.1 μg of DNA/well using lipofectamine reagents (Lipofectamine Plus and Lipofectamine™; Invitrogen) according to the manufacturer's instructions. After incubation of plates for 48 hrs at 37° C., the cells were washed once with warm Dulbecco's Modified Eagle's Medium (D-MEM) without phenol red (Invitrogen) supplemented with 25 mM Hepes (Invitrogen). Transfected HEK-293 cells were treated with EGF for 15 mins, and inhibitors for 1 h (PD98059 and U0126) or 4 hrs (genistein). To each well, 10 μl of DeepBlueC™ coelenterazine substrate (final concentration of 5 μM; PerkinElmer Life Sciences) diluted in Dulbecco's PBS containing 0.1% (w/v) CaCl₂, 0.1% (w/v) D-Glucose, 0.1% (w/v) MgCl₂, and 10 μg/ml aprotinin, was added. The plates were immediately counted using the Fusion Universal Microplate Analyzer (PerkinElmer Life Sciences).

Bioluminescence resulting from Rluc emission was counted at 410 nm using a 370-450 nm band pass filter and the fluorescence of GFP2 was counted at 515 nm using a 500-530 nm band pass filter. The efficiency of energy transfer between Rluc and GFP2 is determined by dividing acceptor emission intensity (GFP2) by donor emission intensity (Rluc). The resulting value reflects the proximity of GFP2 to Rluc and is referred to as the BRET ratio.

### Western Blots and Immunoprecipitation

Near-confluent cultures of HEK-293 cells, stably transfected with either an acceptor-insert-donor construct, or CHIM construct (SEQ ID NO: 2); or ND7 cells were harvested with trypsin-EDTA and resuspended to a density of 6×10⁵ cells/ml. Aliquots (5 ml) of cell suspensions were added to 100 mm cell culture dishes (Corning Incorporated Life Sciences, Acton, Mass.) and incubated for 12 hrs at 37° C. Stably transfected HEK-293 cells were treated with genistein for 4 hrs at 37° C; and ND7 cells were treated with 100 μM PD98059 for 1 h and with 100 ng/ml NGF for 15 min. Plates were washed with 5 ml of ice-cold PBS, and then cells were lysed with modified radioimmunoprecipitation (RIPA) buffer (1% (w/w) IGEPAL CA-630, 1% (w/v) sodium deoxycholate, 0.1% (w/v) SDS, 0.15 M NaCl, 0.01 M sodium phosphate, pH 7.2, 2 mM EDTA, 50 mM sodium fluoride, 1 mM phenylmethylsulfonyl fluoride, sodium vanadate, sodium fluoride, and benzamidine, 10 μg/ml aprotinin, 1 μg/ml leupeptin and pepstatin) at 4° C. for 20 min and centrifuged at 15,000 g for 15 min at 4° C. The protein concentrations of the supernatants were determined by using the BCA (bicinchoninic acid) protein assay (Pierce Chemical Company, Rockford, Ill.). The cell lysates were precleared with immobilized protein A (Pierce Chemical Company) for 3 hrs at 4° C. Immunoprecipitates were carried out with a rabbit anti-GFP or a rabbit anti-L1 polyclonal Ab and immobilized protein A overnight at 4° C. Immobilized protein A beads were washed and resuspended in Laemnuli buffer, analyzed by SDS-PAGE, and transferred to nitrocellulose membrane. The membrane was blocked, washed and then incubated with 1 μg/ml of anti-phosphotyrosine Ab overnight at 4° C. The blot was then incubated with HRP-conjugated goat anti-rabbit Ab at a dilution of 1:5000, and then developed using the enhanced chemiluminescence system (SuperSignal, West Pico chemiluminescent substrate; Pierce Chemical Company). Membranes were stripped using 0.2 M glycine-HCl, (pH 2.5) and reprobed with 0.5 μg/ml of anti-GFP Ab for 2 hrs at room temperature or 2 μg/ml of anti-L1 Ab overnight at 4° C.

### Results and Discussion

A portion of the ankyrin binding domain of L1-CAM, with amino acid sequence QFNEDGSFIGQY (SEQ ID NO: 1), was inserted between Rluc and GFP2 coding regions (FIG. 1A; L1-BRET). As energy transfer depends on the proximity and orientation of the donor and acceptor, the construct was designed to observe conformational changes in the ankyrin-binding domain that accompany tyrosine phosphorylation. A CHIM construct (SEQ ID NO: 2), lacking the L1-CAM sequence, was also generated as a positive control (FIG. 1A).

Stimulation of cells with 100 ng/ml EGF resulted in a significant 24% decrease in the BRET ratio of the L1-BRET construct expressed in HEK293 cells (P<0.01, FIG. 1B, results shown are mean±standard deviation, n=5). In contrast, there was no change in the BRET ratio in similarly-treated cells transfected with the control CHIM construct (SEQ ID NO: 2). Subsequent results were normalized against values obtained from cells transfected with CHIM (SEQ ID NO: 2). Trials using longer inserts (25 aa) showed a similar response to EGF, though of lower amplitude.

To confirm that decreases in the BRET ratio are due to tyrosine phosphorylation, the tyrosine was mutated to an aspartate (QFNEDGSFIGQD, SEQ ID NO: 3), histidine (QFNEDGSFIGQH, SEQ ID NO: 4), or phenylalanine (QFNEDGSFIGQF, SEQ ID NO: 5) residue. These constructs displayed no significant change in the BRET ratio following stimulation of transfected HEK-293 cells with 100 ng/ml EGF (FIG. 1C, results shown are mean±standard deviation, n=5). Interestingly, the relative BRET ratios for the QFNEDGSFIGQD construct were significantly lower (P<0.01) than that observed for the QFNEDGSFIGQF construct in either untreated cells or cells stimulated with EGF. These results support the hypothesis that decreases in the BRET ratio of L1-BRET construct are governed by changes in charge caused by tyrosine phosphorylation of the L1-CAM insert.

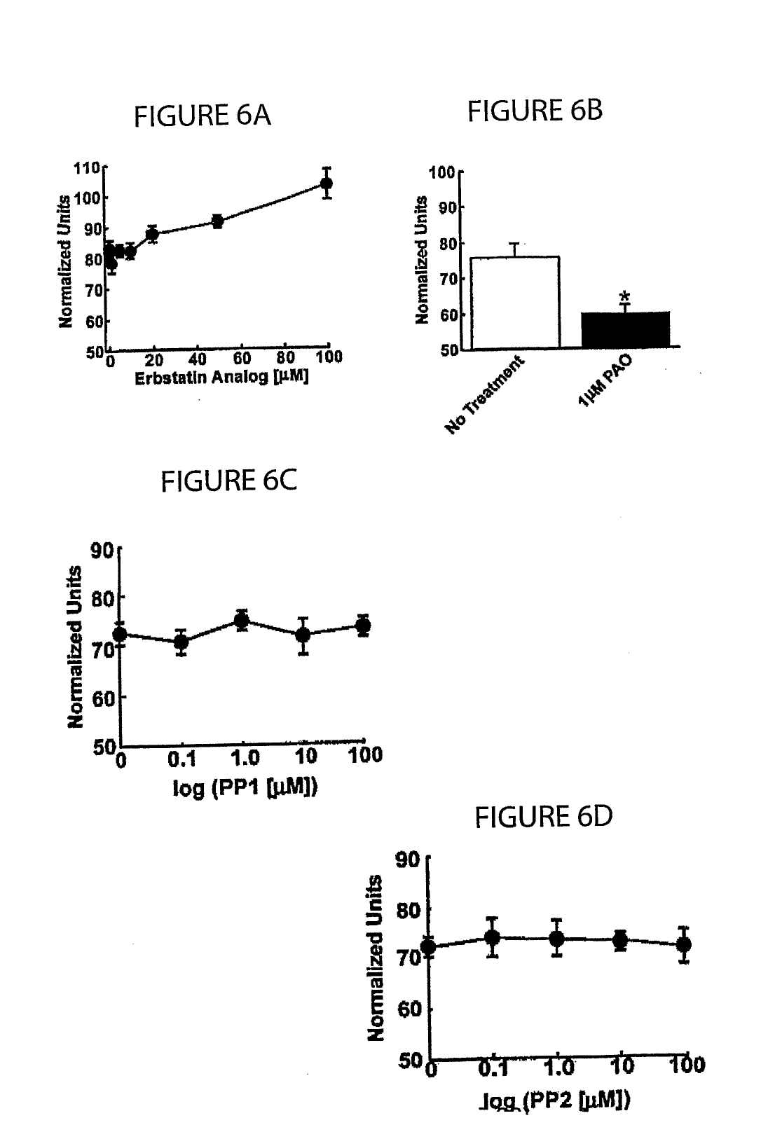

EGF stimulation reduced the BRET ratio of L1-BRET-transfected cells in a dose dependent manner (10-20 ng/ml EGF producing near-maximal reductions; FIG. 1D, results shown are mean±standard deviation, n=5). In EGF-stimulated cells, the reduction in BRET ratio was maximal at 10 min (FIG. 1E, results shown are mean±standard deviation, n=5) and recovered within 60 min, consistent with the transient nature of EGF receptor (EGF-R) signaling events (Marshall (1995) Cell 80:179-185). Phosphotyrosine immunoblots revealed that EGF-R was activated following stimulation of HEK-293 cells with EGF, but not when cells were either serum starved or maintained in medium containing 10% (v/v) fetal bovine serum (FBS). The decrease in BRET ratio following EGF stimulation was reversed by pre-treating cells with genistein, a broad-spectrum tyrosine kinase inhibitor (100 μM, FIG. 1F, results shown are mean±standard deviation, n=5; (Akiyama et al. (1987) J. Biol. Chem. 262:5592-5595)), raising the ratio to a level indistinguishable from that of CHIM (SEQ ID NO: 2). The negative control for genistein, daidzein, had no effect. Inhibiting the EGF receptor-associated kinase with the erbstatin analog (methyl 2,5-dihydroxycinnamate (Umezawa et al. (1990) FEBS Lett. 260:198-200) increased the BRET ratio of the L1-BRET construct in a dose-dependent manner (FIG. 6A, results shown are mean±standard deviation, n=5). Conversely, treatment of transfected HEK-293 cells with phenylarsine oxide, a tyrosine phosphatase inhibitor (Garcia-Morales et al. (1990) Proc. Natl. Acad. Sci. USA 87:9255-9259), resulted in a significant decrease in the BRET ratio (P<0.01; FIG. 6B, results shown are mean±standard deviation, n=5).

Immunoblot analysis was performed (FIG. 1G). Cell lysates were immunoprecipitated with an anti-GFP Ab and subsequently analyzed by immunoblotting with an anti-phosphotyrosine Ab to detect phosphorylated BRET constructs (upper blot). The expression levels of the two constructs are shown in the lower blot. The L1-BRET protein was tyrosine phosphorylated in HEK-293 cells under basal conditions, and completely inhibited when cells were pretreated with genistein (100 μM; FIG. 1G). There was no phosphotyrosine detected in the CHIM construct (SEQ ID NO: 2) in the presence or absence of genistein, consistent with the idea that phosphorylation of the FIGQY (SEQ ID NO: 12) tyrosine is responsible for the changes observed in the spectrum of L1-BRET.

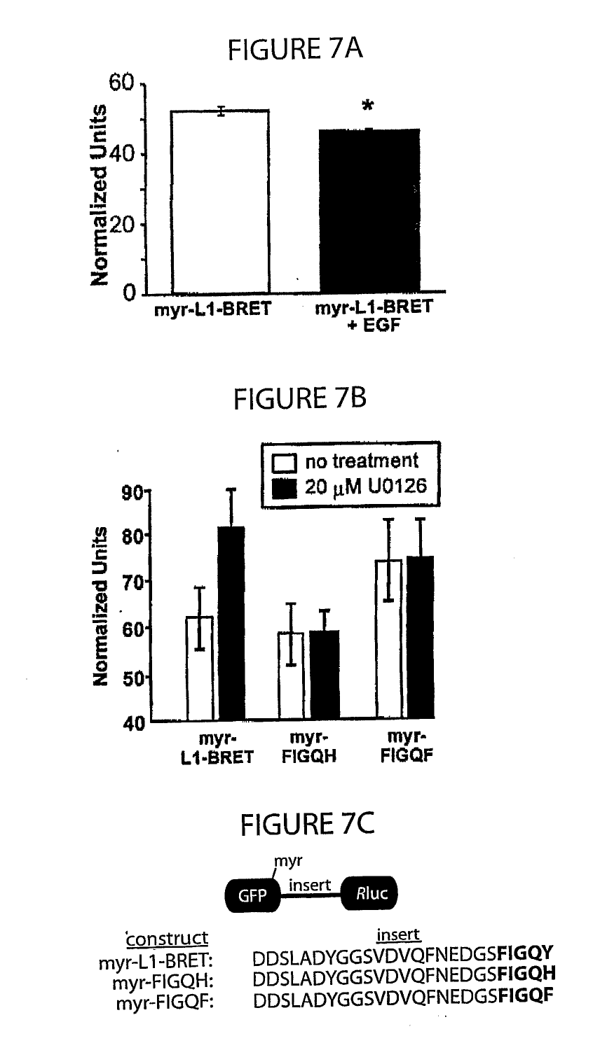

To address the role of membrane localization (see also Example 3), myristoylated constructs were generated (with inserts of SEQ ID NO: 7, SEQ ID NO: 8, or SEQ ID NO: 9), including 25 residues of the L1 cytoplasmic sequence (FIG. 7C; myr-L1-BRET). This construct displayed localization to the plasma membrane when expressed in HEK-293 cells. Like L1-BRET, there was a significant decrease in BRET ratio of the myristoylated construct when HEK-293 cells were stimulated with 100 ng/ml EGF (P<0.01; FIG. 7A, results shown are mean±standard deviation, n=5). These results suggest that growth factor dependent phosphorylation of the FIGQY (SEQ ID NO: 12) sequence was not contingent on its localization to the plasma membrane. Taken together, these results suggest strongly that EGF-R-regulated phosphorylation of L1-BRET depends on tyrosine kinase activity and is inversely related to the BRET ratio of the reporter (FIG. 1H).

FIG. 7B shows that the myr constructs behave in the same manner as do the non-myristoylated constructs (results shown are mean±standard deviation, n=5, 20 μM U0126). Membrane localization is not important fir the interaction between the kinase and its substrate.

### Example 2

### MAPK Pathway Reporter

Constructs for the inserts, BRET assays, Western blots, and immunoprecipitation used in this Example were designed as for Example 1.

### Results and Discussion

Previous work has shown that components of the MAPK pathway, ERK and p90rsk, can phosphorylate directly different serines located in the cytoplasmic domain of L1-CAM (Schaefer et al. (1999) J. Biol. Chem. 274:37965-37973). To investigate whether the MAPK signaling cascade is required for the phosphorylation of the FIGQY (SEQ ID NO: 12) sequence, the effect of two inhibitors of MEK1/2, PD98059 and U0126 (English et al. (2002) Trends Phat-niacol. Sci. 23:40-45), was examined on the BRET ratio of the L1-BRET construct transfected in HEK-293 cells. Both of the MEK inhibitors increased the BRET ratio of the L1-BRET construct in a dose-dependent manner (FIG. 2A and FIG. 2B, results shown are mean±standard deviation, n=5), suggesting that phosphorylation of the FIGQY sequence (SEQ ID NO: 12) is dependent on activation of the MAPK signaling pathway in HEK-293 cells. Strikingly, inhibitors of src-family kinases including PP1 and PP2 (Hanke et al. (1996) J. Biol. Chem. 271:695-701) had no effect on the BRET ratio of EGF-stimulated cells, suggesting that these non-receptor tyrosine kinases do not play a role in the phosphorylation of L1-BRET (FIG. 6C and FIG. 6D, results shown are mean±standard deviation, n=5).

To confirm that the effects of MEK inhibitors were due to the phosphorylation state of QFNEDGSFIGQY (SEQ ID NO: 1), the effect of PD98059 and U0126 was examined on the BRET ratio of the QFNEDGSFIGQD (SEQ ID NO: 3), QFNEDGSFIGQH (SEQ ID NO: 4), and QFNEDGSFIGQF (SEQ ID NO: 5) variant constructs. There was no change in the BRET ratio of the variant constructs following inhibition of transfected HEK-293 cells with MEK inhibitors (100 μM; FIG. 2C, results shown are mean±standard deviation, n=5). To determine whether components of the MAPK cascade are responsible for phosphorylating the FIGQY (SEQ ID NO: 12) sequence in other cell types, downstream of other RTKs, L1-BRET construct was transiently transfected into ND7 (neuroblastoma) and PC12 (pheochromocytoma) cells (Dunn et al. (1991) Brain Res. 545:80-86; Pang et al. (1995) J. Biol. Chem. 270:13585-13588). Activation of the NGF-R resulted in a decrease in the BRET ratio of the L1-BRET construct in ND7 cells, whereas inhibition of tyrosine kinases with genistein resulted in an increase in the BRET ratio. There were also increases in the BRET ratio of the L1-BRET construct when ND7 or PC12 cells were pre-treated with either the PD98059 (FIG. 2D and FIG. 2E, results shown are mean±standard deviation, n=5) or U0126 compounds, suggesting that a common signaling pathway is responsible for L1-BRET phosphorylation in different cell types.

To determine whether the MAPK signaling cascade can modulate tyrosine phosphorylation of full-length L1-CAM, the effect of MEK inhibitors was examined on ND7 cells stimulated with NGF. Cell lysates were immunoprecipitated with an anti-L1-CAM Ab and subsequently analyzed by immunoblotting (FIG. 2F) with an anti-phosphotyrosine Ab to detect phosphorylated L1-CAM (upper blot). The same blot was stripped and re-probed with an Ab directed against L1-CAM. Treatment of NGF-stimulated ND7 cells with the PD98059 compound (100 μM) resulted in a marked decrease in the level of tyrosine phosphorylation of endogenous L1-CAM (FIG. 2F). These results suggest that tyrosine phosphorylation of endogenously-expressed L1-CAM is dependent on the MAPK signaling pathway.

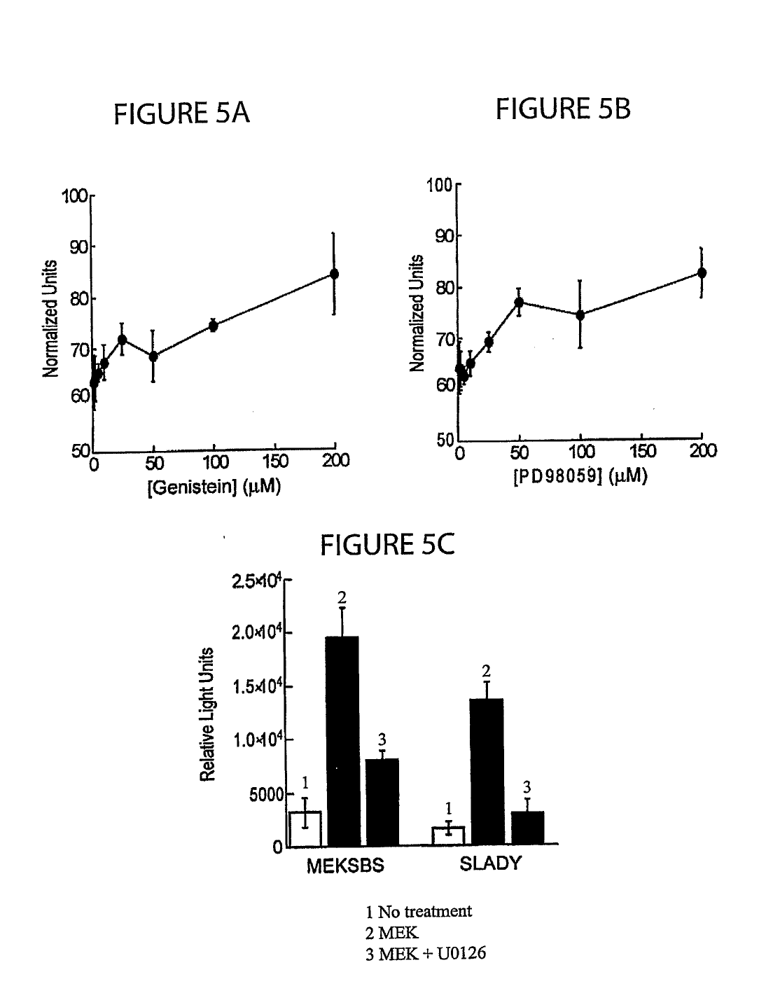

Another MAP kinase pathway reporter was constructed and tested using the methods described above and is outlined in the L1-SLADY BRET Data shown in FIGS. 5A, 5B, and 5C. The results of these figures are consistent with the other MAP kinase pathway data showing that the BRET system can be used as a MAP kinase pathway reporter.

### Example 3

### L1-CAM-Dependent Recruitment to the Plasma Membrane

Construct design for the below particular inserts were performed as above.

### Neurite Outgrowth Assays

Neurite outgrowth experiments were performed as described (Gil et al. (2003) J. Cell Biol. 162:719-30) with slight modification. A 1 cm-diameter circle in a 35 mm petri dish (Becton Dickinson) was coated with poly-L-lysine (5 μg/ml in phosphate-buffered saline (PBS) from Speciality Media, Phillipsburg, N.J.) for 1 hr at room temperature. After several washes with PBS, the coated area was dried under the hood. Aliquots of 1 μl of neural-glial cell adhesion molecule (Ng-CAM, Gil et al. (2003) J. Cell Biol. 162:719-30) at 50 μg/ml or laminin at 30 μg/ml (Becton Dickinson) were spotted on the coated area. Dishes were incubated for 1 hr at room temperature, washed several times with PBS and then blocked with 1% (w/v) bovine serum albumin (BSA). Cerebellar cells were prepared from P2-P4 mouse and plated on the prepared dishes in BME/B27/glucose/glutamine/Pen-Strep media at a cell density of 3×10⁵ cells/ml. Peptides and U0126 were diluted in DMSO (10 mg/ml for peptides, 13 mM for U0126) and further diluted in media (final concentration 10 μg/ml peptide; 10 μM U0126) which was added to the cultures when the cells were plated. Cultures were incubated for 2 days and fixed with 4% paraformaldehyde.

### Immunofluorescence

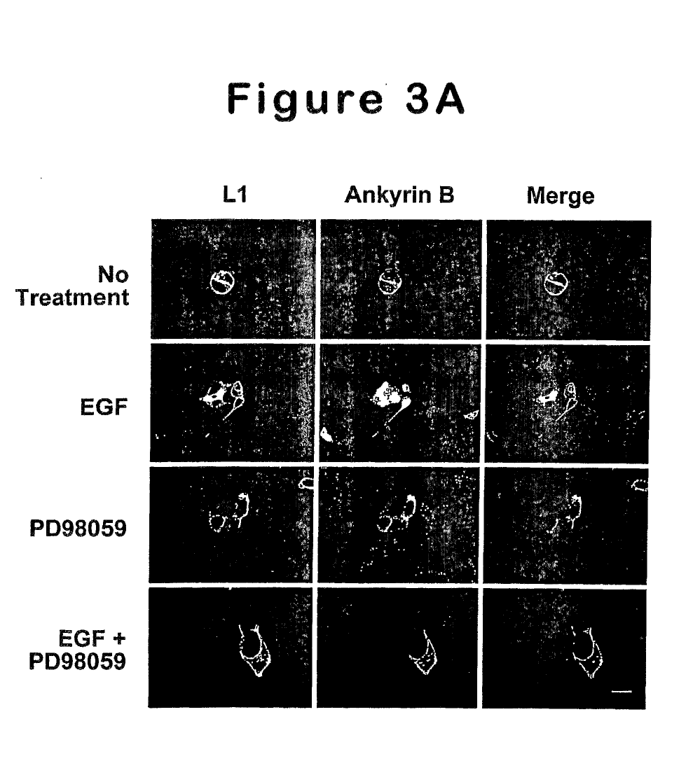

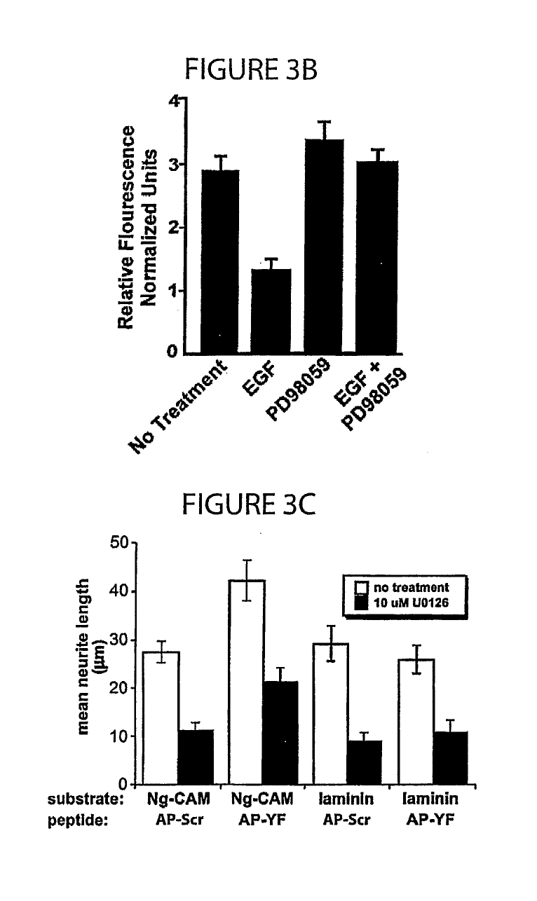

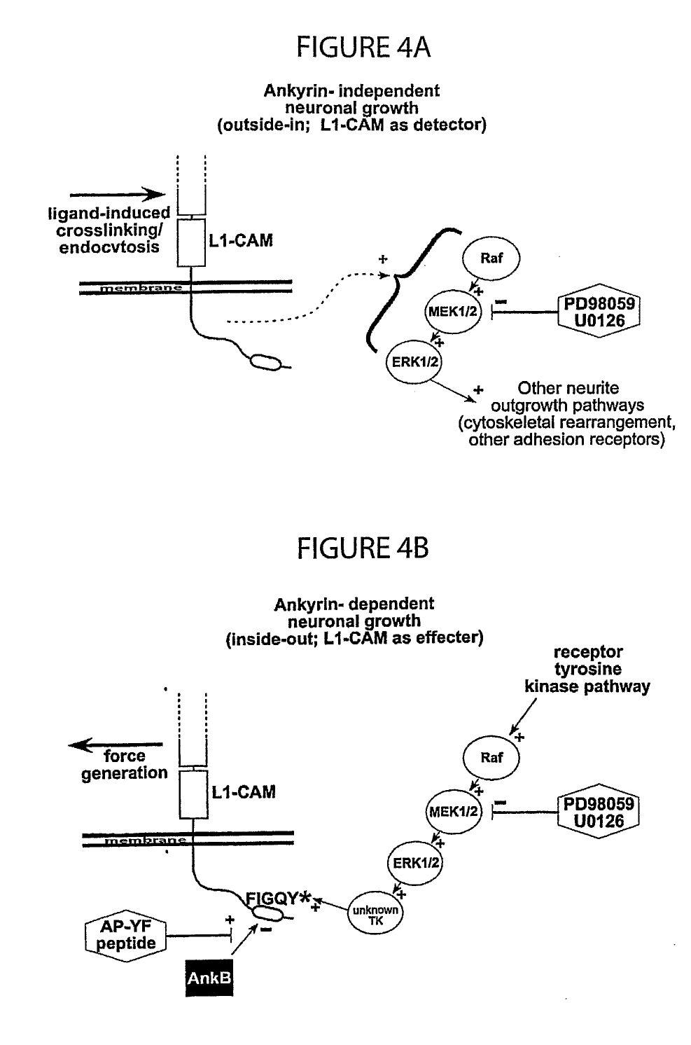

HEK-293 cells were transfected with cDNA encoding an amino-terminal myc-epitope tagged full-length wild-type neuronal adhesion protein L1-CAM and a carboxy-terminal GFP-tagged ankyrin B construct using lipofectamine reagents. Transiently transfected HEK-293 cells were treated for 1 h with 100 μM PD98059 and 100 ng/ml EGF. For immunolocalization, cells were fixed for 10 min using 1% (w/v) paraformaldehyde in 60 mM Pipes, 25 mM Hepes, 10 mM EGTA, and 2 mM MgCl₂. Staining was performed as described previously (Felsenfeld et al. (1999) Nat. Cell Biol. 1:200-6). Briefly, ankyrin B was detected by indirect immunofluorescence using a rabbit anti-GFP polyclonal Ab and a donkey anti-rabbit Ab conjugated to indodicarbocyanince Cy5. L1-CAM was detected by indirect immunofluorescence using a mouse anti-myc monoclonal Ab and a donkey anti-mouse Ab conjugated to indocarbocyanine Cy3. Confocal micrographs were collected on an Olympus microscope using a 60× objective at a plane intersecting cell-cell junctions. Images were analyzed using NIH ImageJ (National Institutes of Health, Bethesda, Md.). See FIG. 3A. Densitometry was performed using a 5 pixel-wide line scan normal to the interface between two L1-CAM-positive cells. Signal maximum for ankyrin staining at the junction between cells was determined at the position of the maximal L1-CAM staining to ensure that membrane rather than juxtamembrane staining was quantified. Minima were determined from the regions of the line overlapping the cytoplasm of either of the two cells. Membrane localization index was determined using the equation index=max/(max-min) as described (Gil et al. (2003) J. Cell Biol. 162:719-30).

### Results and Discussion

Activation of the EGF-R inhibits L1-CAM-dependent ankyrin B recruitment to the plasma membrane (Gil et al. (2003) J. Cell Biol. 162:719-730). To determine whether the MAPK pathway modulates this interaction, the effects of inhibiting MEK on ankyrin B recruitment to the plasma membrane following EGF stimulation was examined. HEK-293 cells co-transfected with cDNAs encoding full-length myc-tagged L1-CAM and ankyrin-B GFP were treated with 100 ng/ml EGF and/or 100 μM PD98059. L1 and ankyrin B were visualized by indirect immunofluorescence using CY3 and CY5 antibodies, respectively. Fluorescent images were combined to determine colocalization.

Treatment of transfected HEK-293 cells with EGF leads to a decrease in the level of ankyrin B recruited to the plasma membrane (FIG. 3A and FIG. 3B, bar in FIG. 3A represents 20 μm, results in FIG. 3B are the mean±standard deviation of two experiments; Gil et al.(2003) J. Cell Biol. 162:719-730). The decrease in the level of ankyrin B recruitment to the plasma membrane after EGF stimulation was reversed following the addition of PD98059 (FIG. 3A and FIG. 3B). These results suggest that components of the MAPK pathway are required for phosphorylating the FIGQY (SEQ ID NO: 12) tyrosine, and as a consequence can modulate the membrane recruitment of ankyrin B. To examine directly the role of MAP kinase signaling in the regulation of L1-CAM function in situ, cerebellar granular neurons were cultured on substrates coated with the L1 ligand Ng-CAM, a chick L1-CAM homolog. As MAP kinase inhibitors block neuronal growth through both L1-CAM and other receptor families, MAP kinase activity has been suggested to regulate pathways common to nerve growth in general. To determine if L1-CAM function was itself modulated by MAP kinase activity, neurons were grown in the presence of both a MEK kinase inhibitor (U0126) and a peptide AP-YF that inhibits L1-CAM interactions with ankyrin. Previous work has demonstrated that AP-YF stimulates L1-dependent neuronal growth (Gil et al. (2003) J. Cell Biol. 162:719-730). AP-YF sequence is based on the L1-BRET domain, suggesting that it serves as a competitive inhibitor of L1-ankyrin interactions. Therefore, it was hypothesized that if MAP kinase lies upstream of L1 phosphorylation, the addition of AP-YF should override the effects of U0126, as AP-YF activity should not depend on the phosphorylation state of L1-CAM. As shown previously, the addition of AP-YF stimulates significantly L1-mediated neuronal growth as compared to a scrambled, control peptide (AP-scramble; FIG. 3C; mean neuronal length). Addition of U0126 reduces mean neurite length. However, in the presence of AP-YF, neuronal growth was stimulated by almost two fold as compared to neurons grown in the presence of a control peptide. Axon growth on laminin was inhibited by U0126 but was not rescued by AP-YF treatment. These results strongly suggest that MAP kinase activity regulates Ll-CAM-mediated neuronal growth in an ankyrin dependent manner.

### Example 4

### Src Pathway BRET Reporter

Construct design for the below particular inserts and BRET assays was as in Example 1.

### Results and Discussion

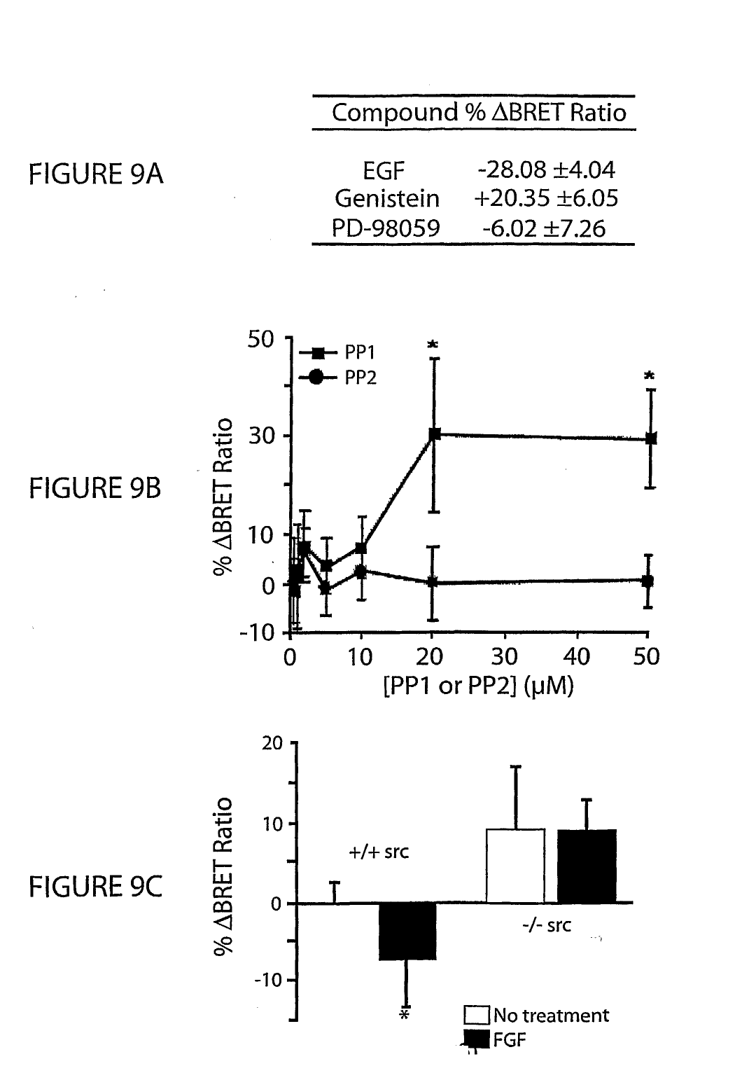

Using a target sequence derived from a tyrosine in the L1-CAM cytoplasmic tail, a reporter was generated encoding a 12 amino acid insert, including a terminal tyrosine (LCFIKRSKGGKY, SEQ ID NO: 13). Like the MEK1/2 reporter, this construct displays an EGF-dependent decrease in BRET efficiency which is reversed by the addition of the tyrosine kinase inhibitor genistein (100 μM genistein, 100 ng/ml EGF; FIG. 9A, results are shown mean±standard deviation, *P<0.01). The MEK1/2 inhibitor PD98059 (100 μM) has no detectible effect on this reporter. However, the Src-family inhibitor PP1 causes an increase in the BRET efficiency at doses above 20 μM while a related inhibitor PP2 has no effect at similar doses (FIG. 9B, results are shown mean±standard deviation, *P<0.01). The selectivity of these related inhibitors suggests that this reporter is selective for Src itself, an observation supported by the use of Src-deficient fibroblasts. Fibroblasts derived from wild-type (+/+src) or Src-null (−/−src) mice were treated with FGF, which activates indirectly Src-family kinases. Wild-type fibroblasts display an FGF-dependent decrease in BRET efficiency similar to that seen in HEK-293 cells with EGF. In the absence of Src, FGF has no effect on energy transfer and the base-line BRET efficiency is elevated, consistent with an overall reduction of phosphorylation of the construct. This shows fibroblasts have no response to growth factor agonists of membrane-linked tyrosine kinase receptors (FIG. 9C). Together, these results strongly suggest that this reporter, based on a sequence derived from the L1-CAM cytoplasmic tail serves as a reporter for Src kinase activity.

### Example 5

### BRET Probe for Protein Kinase A

Constructs for the particular inserts and BRET assays in this Example 5 were designed as in Example 1 above.

### Results and Discussion

A reporter was generated for PKA based on a target domain derived from fish connexin35 (Mitropoulou et al. (2003) J. Neurosci. Res. 72:147-157). The PKA insert target sequence (QSAKQKERRYS) contains a carboxy-terminal serine phosphorylation target.

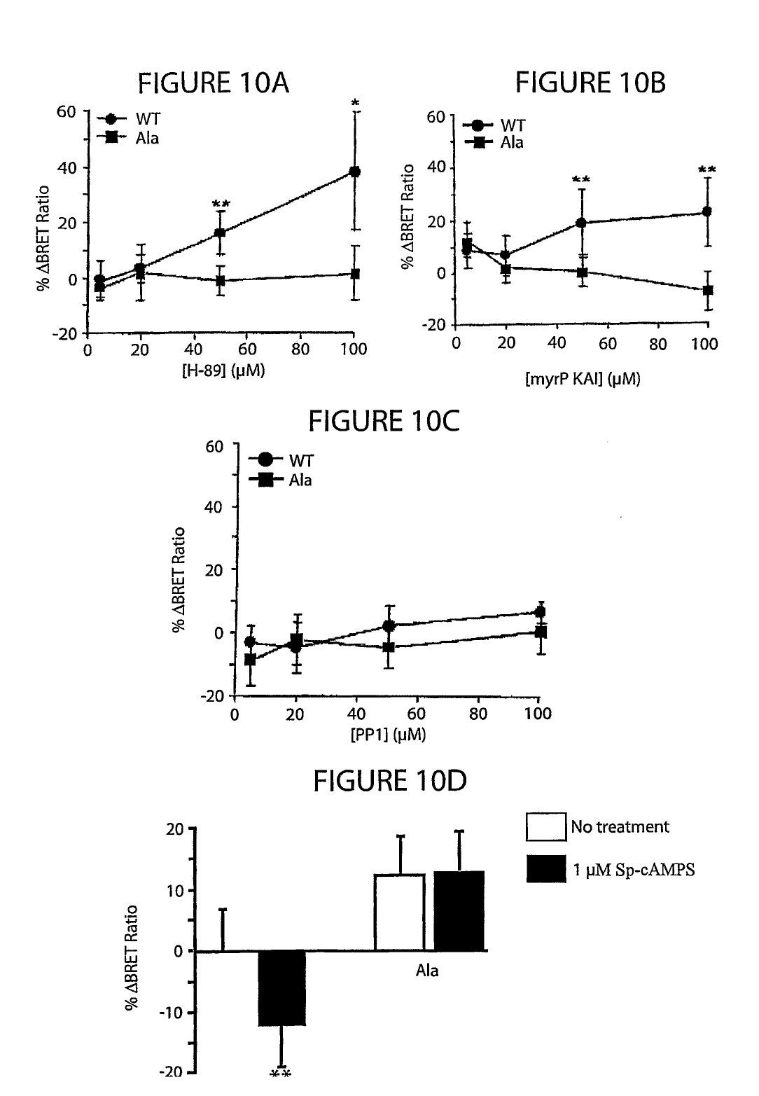

As in the case of the MEK1/2 reporter, the PKA reporter showed a decrease in BRET efficiency under conditions that stimulate PKA activity including treatment of cells with non-hydrolysable cAMP analogs (Sp-cAMPs (Adenosine-3′,5′-cyclic monophosphorothioate, Sp-isomer); FIG. 10D, results are shown mean±standard deviation, *P<0.01, **P<0.05). PP1, an inhibitor of src-family kinases had no effect on FRET efficiency (FIG. 10C, results are shown mean±standard deviation, *P<0.01, **P<0.05). However, PKA inhibitors, including H89 and myrPKAI caused a dose-dependent increase in FRET efficiency (FIG. 10A and FIG. 10B, results are shown mean±standard deviation, *P<0.01, **P<0.05), consistent with the idea that the BRET reporter system functions through a phosphorylation-dependent conformational change, perhaps a result of a charge repulsion between the added phosphate group and the downstream luciferase. Together, these results suggest that the BRET reporter is a reliable indicator of PKA activity. Additionally, these results demonstrate that the construct design can be applied to both tyrosine and serine/threonine kinases.

### Example 6

### Optimal Location for Post-Translational Modification

Constructs for the inserts and BRET assays used in this Example were designed as for Example 1.

### Results and Discussion

FIG. 8 shows that the position of the tyrosine within the insert used to detect phosphorylation is important. The SACT-A sequence (QFNEDGSFIGQY, SEQ ID NO: 1) positions the tyrosine at the C-terminal end of the insert. This is not the case in SACT-B (NEDGSFIGQYSG, SEQ ID NO: 10) or SACT-C (DGSFIGQYSGKK, SEQ ID NO: 11). The data show that upon treatment of EGF, which induces phosphorylation of the tyrosine in the FIGQY sequence, the SACT-A BRET ratio changes, whereas the SACT-B and SACT-C BRET ratios do not change and changes much less significantly, respectively. Therefore, the tyrosine that undergoes phosphorylation is optimally at the C-terminal position of the insert.

Numerous references, including patents, patent applications and various publications, are cited and discussed in the description of this invention. The citation and/or discussion of such references is provided merely to clarify the description of the present invention and is not an admission that any such reference is “prior art” to the invention described here. All references cited and/or discussed in this specification are incorporated herein by reference in their entirety and to the same extent as if each reference was individually incorporated by reference.

SEQUENCES
SEQ ID NO: 1 (amino acid - natural - human)
QFNEDGSFIGQY
SEQ ID NO: 2 (nucleic acid - synthetic - plasmid)
GACGGATCGGGAGATCTCCCGATCCCCTATGGTGCACTCTCAGTACAATC
TGCTCTGATGCCGCATAGTT
AAGCCAGTATCTGCTCCCTGCTTGTGTGTTGGAGGTCGCTGAGTAGTGCG
CGAGCAAAATTTAAGCTACA
ACAAGGCAAGGCTTGACCGACAATTGCATGAAGAATCTGCTTAGGGTTAG
GCGTTTTGCGCTGCTTCGCG
ATGTACGGGCCAGATATACGCGTTGACATTGATTATTGACTAGTTATTAA
TAGTAATCAATTACGGGGTC
ATTAGTTCATAGCCCATATATGGAGTTCCGCGTTACATAACTTACGGTAA
ATGGCCCGCCTGGCTGACCG
CCCAACGACCCCCGCCCATTGACGTCAATAATGACGTATGTTCCCATAGT
AACGCCAATAGGGACTTTCC
ATTGACGTCAATGGGTGGAGTATTTACGGTAAACTGCCCACTTGGCAGTA
CATCAAGTGTATCATATGCC
AAGTACGCCCCCTATTGACGTCAATGACGGTAAATGGCCCGCCTGGCATT
ATGCCCAGTACATGACCTTA
TGGGACTTTCCTACTTGGCAGTACATCTACGTATTAGTCATCGCTATTAC
CATGGTGATGCGGTTTTGGC
AGTACATCAATGGGCGTGGATAGCGGTTTGACTCACGGGGATTTCCAAGT
CTCCACCCCATTGACGTCAA
TGGGAGTTTGTTTTGGCACCAAAATCAACGGGACTTTCCAAAATGTCGTA
ACAACTCCGCCCCATTGACG
CAAATGGGCGGTAGGCGTGTACGGTGGGAGGTCTATATAAGCAGAGCTCT
CTGGCTAACTAGAGAACCCA
CTGCTTACTGGCTTATCGAAATTAATACGACTCACTATAGGGAGACCCAA
GCTGGCTAGCGTTTAAACTT
AAGCTTGGTACCGAGCTCGGATCCACTAGTCCAGTGTGGTGGAATTCTGC
AGATATCCAGCACAGTGGCG
GCCGCAATGGTGAGCAAGGGCGAGGAGCTGTTCACCGGGGTGGTGCCCAT
CCTGGTCGAGCTGGACGGCG
ACGTAAACGGCCACAAGTTCAGCGTGTCCGGCGAGGGCGAGGGCGATGCC
ACCTACGGCAAGCTGACCCT
GAAGTTCATCTGCACCACCGGCAAGCTGCCCGTGCCCTGGCCCACCCTCG
TGACCACCCTGAGCTACGGC
GTGCAGTGCTTCAGCCGCTACCCCGACCACATGAAGCAGCACGACTTCTT
CAAGTCCGCCATGCCCGAAG
GCTACGTCCAGGAGCGCACCATCTTCTTCAAGGACGACGGCAACTACAAG
ACCCGCGCCGAGGTGAAGTT
CGAGGGCGACACCCTGGTGAACCGCATCGAGCTGAAGGGCATCGACTTCA
AGGAGGACGGCAACATCCTG
GGGCACAAGCTGGAGTACAACTACAACAGCCACAACGTCTATATCATGGC
CGACAAGCAGAAGAACGGCA
TCAAGGTGAACTTCAAGATCCGCCACAACATCGAGGACGGCAGCGTGCAG
CTCGCCGACCACTACCAGCA
GAACACCCCCATCGGCGACGGCCCCGTGCTGCTGCCCGACAACCACTACC
TGAGCACCCAGTCCGCCCTG
AGCAAAGACCCCAACGAGAAGCGCGATCACATGGTCCTGCTGGAGTTCGT
GACCGCCGCCGGGATCACTC
TCGGCATGGACGAGCTGTACAAGTCCGGAGGCGCGCCAATGACCAGCAAG
GTGTACGACCCCGAGCAGAG
GAAGAGGATGATCACCGGCCCCCAGTGGTGGGCCAGGTGCAAGCAGATGA
ACGTGCTGGACAGCTTCATC
AACTACTACGACAGCGAGAAGCACGCCGAGAACGCCGTGATCTTCCTGCA
CGGCAACGCCGCTAGCAGCT
ACCTGTGGAGGCACGTGGTGCCCCACATCGAGCCCGTGGCCAGGTGCATC
ATCCCCGATCTGATCGGCAT
GGGCAAGAGCGGCAAGAGCGGCAACGGCAGCTACAGGCTGCTGGACCACT
ACAAGTACCTGACCGCCTGG
TTCGAGCTCCTGAACCTGCCCAAGAAGATCATCTTCGTGGGCCACGACTG
GGGCGCCTGCCTGGCCTTCC
ACTACAGCTACGAGCACCAGGACAAGATCAAGGCCATCGTGCACGCCGAG
AGCGTGGTGGACGTGATCGA
GAGCTGGGACGAGTGGCCAGACATCGAGGAGGACATCGCCCTGATCAAGA
GCGAGGAGGGCGAGAAGATG
GTGCTGGAGAACAACTTCTTCGTGGAGACCATGCTGCCCAGCAAGATCAT
GAGAAAGCTGGAGCCCGAGG
AGTTCGCCGCCTACCTGGAGCCCTTCAAGGAGAAGGGCGAGGTGAGAAGA
CCCACCCTGAGCTGGCCCAG
AGAGATCCCCCTGGTGAAGGGCGGCAAGCCCGACGTGGTGCAGATCGTGA
GAAACTACAACGCCTACCTG
AGAGCCAGCGACGACCTGCCCAAGATGTTCATCGAGAGCGACCCCGGCTT
CTTCAGCAACGCCATCGTGG
AGGGCGCCAAGAAGTTCCCCAACACCGAGTTCGTGAAGGTGAAGGGCCTG
CACTTCAGCCAGGAGGACGC
CCCCGACGAGATGGGCAAGTACATCAAGAGCTTCGTGGAGAGAGTGCTGA
AGAACGAGCAGTAAGAATTC
GATCTAGACTCGAGTCTAGAGGGCCCGTTTAAACCCGCTGATCAGCCTCG
ACTGTGCCTTCTAGTTGCCA
GCCATCTGTTGTTTGCCCCTCCCCCGTGCCTTCCTTGACCCTGGAAGGTG
CCACTCCCACTGTCCTTTCC
TAATAAAATGAGGAAATTGCATCGCATTGTCTGAGTAGGTGTCATTCTAT
TCTGGGGGGTGGGGTGGGGC
AGGACAGCAAGGGGGAGGATTGGGAAGACAATAGCAGGCATGCTGGGGAT
GCGGTGGGCTCTATGGCTTC
TGAGGCGGAAAGAACCAGCTGGGGCTCTAGGGGGTATCCCCACGCGCCCT
GTAGCGGCGCATTAAGCGCG
GCGGGTGTGGTGGTTACGCGCAGCGTGACCGCTACACTTGCCAGCGCCCT
AGCGCCCGCTCCTTTCGCTT
TCTTCCCTTCCTTTCTCGCCACGTTCGCCGGCTTTCCCCGTCAAGCTCTA
AATCGGGGGCTCCCTTTAGG
GTTCCGATTTAGTGCTTTACGGCACCTCGACCCCAAAAAACTTGATTAGG
GTGATGGTTCACGTAGTGGG
CCATCGCCCTGATAGACGGTTTTTCGCCCTTTGACGTTGGAGTCCACGTT
CTTTAATAGTGGACTCTTGT
TCCAAACTGGAACAACACTCAACCCTATCTCGGTCTATTCTTTTGATTTA
TAAGGGATTTTGCCGATTTC
GGCCTATTGGTTAAAAAATGAGCTGATTTAACAAAAATTTAACGCGAATT
AATTCTGTGGAATGTGTGTC
AGTTAGGGTGTGGAAAGTCCCCAGGCTCCCCAGCAGGCAGAAGTATGCAA
AGCATGCATCTCAATTAGTC
AGCAACCAGGTGTGGAAAGTCCCCAGGCTCCCCAGCAGGCAGAAGTATGC
AAAGCATGCATCTCAATTAG
TCAGCAACCATAGTCCCGCCCCTAACTCCGCCCATCCCGCCCCTAACTCC
GCCCAGTTCCGCCCATTCTC
CGCCCCATGGCTGACTAATTTTTTTTATTTATGCAGAGGCCGAGGCCGCC
TCTGCCTCTGAGCTATTCCA
GAAGTAGTGAGGAGGCTTTTTTGGAGGCCTAGGCTTTTGCAAAAAGCTCC
CGGGAGCTTGTATATCCATT
TTCGGATCTGATCAGCACGTGATGAAAAAGCCTGAACTCACCGCGACGTC
TGTCGAGAAGTTTCTGATCG
AAAAGTTCGACAGCGTCTCCGACCTGATGCAGCTCTCGGAGGGCGAAGAA
TCTCGTGCTTTCAGCTTCGA
TGTAGGAGGGCGTGGATATGTCCTGCGGGTAAATAGCTGCGCCGATGGTT
TCTACAAAGATCGTTATGTT
TATCGGCACTTTGCATCGGCCGCGCTCCCGATTCCGGAAGTGCTTGACAT
TGGGGAATTCAGCGAGAGCC
TGACCTATTGCATCTCCCGCCGTGCACAGGGTGTCACGTTGCAAGACCTG
CCTGAAACCGAACTGCCCGC
TGTTCTGCAGCCGGTCGCGGAGGCCATGGATGCGATCGCTGCGGCCGATC
TTAGCCAGACGAGCGGGTTC
GGCCCATTCGGACCGCAAGGAATCGGTCAATACACTACATGGCGTGATTT
CATATGCGCGATTGCTGATC
CCCATGTGTATCACTGGCAAACTGTGATGGACGACACCGTCAGTGCGTCC
GTCGCGCAGGCTCTCGATGA
GCTGATGCTTTGGGCCGAGGACTGCCCCGAAGTCCGGCACCTCGTGCACG
CGGATTTCGGCTCCAACAAT
GTCCTGACGGACAATGGCCGCATAACAGCGGTCATTGACTGGAGCGAGGC
GATGTTCGGGGATTCCCAAT
ACGAGGTCGCCAACATCTTCTTCTGGAGGCCGTGGTTGGCTTGTATGGAG
CAGCAGACGCGCTACTTCGA
GCGGAGGCATCCGGAGCTTGCAGGATCGCCGCGGCTCCGGGCGTATATGC
TCCGCATTGGTCTTGACCAA
CTCTATCAGAGCTTGGTTGACGGCAATTTCGATGATGCAGCTTGGGCGCA
GGGTCGATGCGACGCAATCG
TCCGATCCGGAGCCGGGACTGTCGGGCGTACACAAATCGCCCGCAGAAGC
GCGGCCGTCTGGACCGATGG
CTGTGTAGAAGTACTCGCCGATAGTGGAAACCGACGCCCCAGCACTCGTC
CGAGGGCAAAGGAATAGCAC
GTGCTACGAGATTTCGATTCCACCGCCGCCTTCTATGAAAGGTTGGGCTT
CGGAATCGTTTTCCGGGACG
CCGGCTGGATGATCCTCCAGCGCGGGGATCTCATGCTGGAGTTCTTCGCC
CACCCCAACTTGTTTATTGC
AGCTTATAATGGTTACAAATAAAGCAATAGCATCACAAATTTCACAAATA
AAGCATTTTTTTCACTGCAT
TCTAGTTGTGGTTTGTCCAAACTCATCAATGTATCTTATCATGTCTGTAT
ACCGTCGACCTCTAGCTAGA
GCTTGGCGTAATCATGGTCATAGCTGTTTCCTGTGTGAAATTGTTATCCG
CTCACAATTCCACACAACAT
ACGAGCCGGAAGCATAAAGTGTAAAGCCTGGGGTGCCTAATGAGTGAGCT
AACTCACATTAATTGCGTTG
CGCTCACTGCCCGCTTTCCAGTCGGGAAACCTGTCGTGCCAGCTGCATTA
ATGAATCGGCCAACGCGCGG
GGAGAGGCGGTTTGCGTATTGGGCGCTCTTCCGCTTCCTCGCTCACTGAC
TCGCTGCGCTCGGTCGTTCG
GCTGCGGCGAGCGGTATCAGCTCACTCAAAGGCGGTAATACGGTTATCCA
CAGAATCAGGGGATAACGCA
GGAAAGAACATGTGAGCAAAAGGCCAGCAAAAGGCCAGGAACCGTAAAAA
GGCCGCGTTGCTGGCGTTTT
TCCATAGGCTCCGCCCCCCTGACGAGCATCACAAAAATCGACGCTCAAGT
CAGAGGTGGCGAAACCCGAC
AGGACTATAAAGATACCAGGCGTTTCCCCCTGGAAGCTCCCTCGTGCGCT
CTCCTGTTCCGACCCTGCCG
CTTACCGGATACCTGTCCGCCTTTCTCCCTTCGGGAAGCGTGGCGCTTTC
TCATAGCTCACGCTGTAGGT
ATCTCAGTTCGGTGTAGGTCGTTCGCTCCAAGCTGGGCTGTGTGCACGAA
CCCCCCGTTCAGCCCGACCG
CTGCGCCTTATCCGGTAACTATCGTCTTGAGTCCAACCCGGTAAGACACG
ACTTATCGCCACTGGCAGCA
GCCACTGGTAACAGGATTAGCAGAGCGAGGTATGTAGGCGGTGCTACAGA
GTTCTTGAAGTGGTGGCCTA
ACTACGGCTACACTAGAAGAACAGTATTTGGTATCTGCGCTCTGCTGAAG
CCAGTTACCTTCGGAAAAAG
AGTTGGTAGCTCTTGATCCGGCAAACAAACCACCGCTGGTAGCGGTTTTT
TTGTTTGCAAGCAGCAGATT
ACGCGCAGAAAAAAAGGATCTCAAGAAGATCCTTTGATCTTTTCTACGGG
GTCTGACGCTCAGTGGAACG
AAAACTCACGTTAAGGGATTTTGGTCATGAGATTATCAAAAAGGATCTTC
ACCTAGATCCTTTTAAATTA
AAAATGAAGTTTTAAATCAATCTAAAGTATATATGAGTAAACTTGGTCTG
ACAGTTACCAATGCTTAATC
AGTGAGGCACCTATCTCAGCGATCTGTCTATTTCGTTCATCCATAGTTGC
CTGACTCCCCGTCGTGTAGA
TAACTACGATACGGGAGGGCTTACCATCTGGCCCCAGTGCTGCAATGATA
CCGCGAGACCCACGCTCACC
GGCTCCAGATTTATCAGCAATAAACCAGCCAGCCGGAAGGGCCGAGCGCA
GAAGTGGTCCTGCAACTTTA
TCCGCCTCCATCCAGTCTATTAATTGTTGCCGGGAAGCTAGAGTAAGTAG
TTCGCCAGTTAATAGTTTGC
GCAACGTTGTTGCCATTGCTACAGGCATCGTGGTGTCACGCTCGTCGTTT
GGTATGGCTTCATTCAGCTC
CGGTTCCCAACGATCAAGGCGAGTTACATGATCCCCCATGTTGTGCAAAA
AAGCGGTTAGCTCCTTCGGT
CCTCCGATCGTTGTCAGAAGTAAGTTGGCCGCAGTGTTATCACTCATGGT
TATGGCAGCACTGCATAATT
CTCTTACTGTCATGCCATCCGTAAGATGCTTTTCTGTGACTGGTGAGTAC
TCAACCAAGTCATTCTGAGA
ATAGTGTATGCGGCGACCGAGTTGCTCTTGCCCGGCGTCAATACGGGATA
ATACCGCGCCACATAGCAGA
ACTTTAAAAGTGCTCATCATTGGAAAACGTTCTTCGGGGCGAAAACTCTC
AAGGATCTTACCGCTGTTGA
GATCCAGTTCGATGTAACCCACTCGTGCACCCAACTGATCTTCAGCATCT
TTTACTTTCACCAGCGTTTC
TGGGTGAGCAAAAACAGGAAGGCAAAATGCCGCAAAAAAGGGAATAAGGG
CGACACGGAAATGTTGAATA
CTCATACTCTTCCTTTTTCAATATTATTGAAGCATTTATCAGGGTTATTG
TCTCATGAGCGGATACATAT
TTGAATGTATTTAGAAAAATAAACAAATAGGGGTTCCGCGCACATTTCCC
CGAAAAGTGCCACCTGACGTC
SEQ ID NO: 3 (amino acid - artificial)
QFNEDGSFIGQD
SEQ ID NO: 4 (amino acid - artificial)
QFNEDGSFIGQH
SEQ ID NO: 5 (amino acid - artificial)
QFNEDGSFIGQF
SEQ ID NO: 6 (amino acid - natural - human)
KPLGSDDSLADY
SEQ ID NO: 7 (amino acid - natural - human)
DDSLADYGGSVDVQFNEDGSFIGQY
SEQ ID NO: 8 (amino acid - artificial)
DDSLADYGGSVDVQFNEDGSFIGQH
SEQ ID NO: 9 (amino acid - artificial)
DDSLADYGGSVDVQFNEDGSFIGQF
SEQ ID NO: 10 (amino acid - natural - human)
NEDGSFIGQYSG
SEQ ID NO: 11 (amino acid - natural - human)
DGSFIGQYSGKK
SEQ ID NO: 12 (amino acid - natural - human)
FIGQY
SEQ ID NO: 13 (amino acid - natural - human)
LCFIKRSKGGKY
SEQ ID NO: 14 (artificial)
[Biotin]-A-D-P-D-H-D-H-T-G-F-L-T-E-Y-V-A-T-R-W-
[OH]
SEQ NO: 15 (BRETchim1)
MVSKGEELFTGVVPILVELDGDVNGHKFSVSGEGEGDATYGKLTLKFICT
TGKLPVPWPTLVTTLSYGVQCFSRYPDHMKQHDFFKSAMPEGYVQERTIF
FKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNH
YLSTQSALSKDPNEKRDHMVLLEFVTAAGITLGMDELYKSGGAPMTSKVY
DPEQRKRMITGPQWWARCKQMNVLDSFINYYDSEKHAENAVIFLHGNAAS
SYLWRHVVPHIEPVARCIIPDLIGMGKSGKSGNGSYRLLDHYKYLTAWFE
LLNLPKKIIFVGHDWGACLAFHYSYEHQDKIKAIVHAESVVDVIESWDEW
PDIEEDIALIKSEEGEKMVLENNFFVETMLPSKIMRKLEPEEFAAYLEPF
KEKGEVRRPTLSWPREIPLVKGGKPDVVQIVRNYNAYLRASDDLPKMFIE
SDPGFFSNAIVEGAKKFPNTEFVKVKGLHFSQEDAPDEMGKYIKSFVERV
LKNEQ
SEQ ID NO: 16 (FRET.FIGQY)
MVSKGEELFTGVVPILVELDGDVNGHKFSVSGEGEGDATYGKLTLKLICT
TGKLPVPWPTLVTTLGYGLQCFARYPDHMKQHDFFKSAMPEGYVQERTIF
FKDDGNYKTRAEVKFEGDTLVNRIELKGIDFKEDGNILGHKLEYNYNSHN
VYITADKQKNGIKANFKIRHNIEDGGVQLADHYQQNTPIGDGPVLLPDNH
YLSYQSALSKDPNEKRDHMVLLEFVTAAGITLGMDELYKQFNEDGSFIGQ
YGAPMVSKGEELFTGVVPILVELDGDVNGHKFSVSGEGEGDATYGKLTLK
FICTTGKLPVPWPTLVTTLTWGVQCFSRYPDHMKQHDFFKSAMPEGYVQE
RTIFFKDDGNYKTRAEVKFEGDTLVNRIELKGIDFKEDGNILGHKLEYNY
ISHNVYITADKQKNGIKANFKIRHNIEDGSVQLADHYQQNTPIGDGPVLL
PDNHYLSTQSALSKDPNEKRDHMVLLEFVTAAGITLGMDELYK
SEQ ID NO: 17 (FRETchim)
MVSKGEELFTGVVPILVELDGDVNGHKFSVSGEGEGDATYGKLTLKLICT
TGKLPVPWPTLVTTLGYGLQCFARYPDHMKQHDFFKSAMPEGYVQERTIF
FKDDGNYKTRAEVKFEGDTLVNRIELKGIDFKEDGNILGHKLEYNYNSHN
VYITADKQKNGIKANFKIRHNIEDGGVQLADHYQQNTPIGDGPVLLPDNH
YLSYQSALSKDPNEKRDHMVLLEFVTAAGITLGMDELYKSGGAPMVSKGE
ELFTGVVPILVELDGDVNGHKFSVSGEGEGDATYGKLTLKFICTTGKLPV
PWPTLVTTLTWGVQCFSRYPDHMKQHDFFKSAMPEGYVQERTIFFKDDGN
YKTRAEVKFEGDTLVNRIELKGIDFKEDGNILGHKLEYNYISHNVYITAD
KQKNGIKANFKIRHNIEDGSVQLADHYQQNTPIGDGPVLLPDNHYLSTQS
ALSKDPNEKRDHMVLLEFVTAAGITLGMDELYK
SEQ ID NO: 18 (pcBRETchim1.FIGQY)
GACGGATCGGGAGATCTCCCGATCCCCTATGGTGCACTCTCAGTACAATC
TGCTCTGATGCCGCATAGTT
AAGCCAGTATCTGCTCCCTGCTTGTGTGTTGGAGGTCGCTGAGTAGTGCG
CGAGCAAAATTTAAGCTACA
ACAAGGCAAGGCTTGACCGACAATTGCATGAAGAATCTGCTTAGGGTTAG
GCGTTTTGCGCTGCTTCGCG
ATGTACGGGCCAGATATACGCGTTGACATTGATTATTGACTAGTTATTAA
TAGTAATCAATTACGGGGTC
ATTAGTTCATAGCCCATATATGGAGTTCCGCGTTACATAACTTACGGTAA
ATGGCCCGCCTGGCTGACCG
CCCAACGACCCCCGCCCATTGACGTCAATAATGACGTATGTTCCCATAGT
AACGCCAATAGGGACTTTCC
ATTGACGTCAATGGGTGGAGTATTTACGGTAAACTGCCCACTTGGCAGTA
CATCAAGTGTATCATATGCC
AAGTACGCCCCCTATTGACGTCAATGACGGTAAATGGCCCGCCTGGCATT
ATGCCCAGTACATGACCTTA
TGGGACTTTCCTACTTGGCAGTACATCTACGTATTAGTCATCGCTATTAC
CATGGTGATGCGGTTTTGGC
AGTACATCAATGGGCGTGGATAGCGGTTTGACTCACGGGGATTTCCAAGT
CTCCACCCCATTGACGTCAA
TGGGAGTTTGTTTTGGCACCAAAATCAACGGGACTTTCCAAAATGTCGTA
ACAACTCCGCCCCATTGACG
CAAATGGGCGGTAGGCGTGTACGGTGGGAGGTCTATATAAGCAGAGCTCT
CTGGCTAACTAGAGAACCCA
CTGCTTACTGGCTTATCGAAATTAATACGACTCACTATAGGGAGACCCAA
GCTGGCTAGCGTTTAAACTT
AAGCTTGGTACCGAGCTCGGATCCACTAGTCCAGTGTGGTGGAATTCTGC
AGATATCCAGCACAGTGGCG
GCCGCAATGGTGAGCAAGGGCGAGGAGCTGTTCACCGGGGTGGTGCCCAT
CCTGGTCGAGCTGGACGGCG
ACGTAAACGGCCACAAGTTCAGCGTGTCCGGCGAGGGCGAGGGCGATGCC
ACCTACGGCAAGCTGACCCT
GAAGTTCATCTGCACCACCGGCAAGCTGCCCGTGCCCTGGCCCACCCTCG
TGACCACCCTGAGCTACGGC
GTGCAGTGCTTCAGCCGCTACCCCGACCACATGAAGCAGCACGACTTCTT
CAAGTCCGCCATGCCCGAAG
GCTACGTCCAGGAGCGCACCATCTTCTTCAAGGACGACGGCAACTACAAG
ACCCGCGCCGAGGTGAAGTT
CGAGGGCGACACCCTGGTGAACCGCATCGAGCTGAAGGGCATCGACTTCA
AGGAGGACGGCAACATCCTG
GGGCACAAGCTGGAGTACAACTACAACAGCCACAACGTCTATATCATGGC
CGACAAGCAGAAGAACGGCA
TCAAGGTGAACTTCAAGATCCGCCACAACATCGAGGACGGCAGCGTGCAG
CTCGCCGACCACTACCAGCA
GAACACCCCCATCGGCGACGGCCCCGTGCTGCTGCCCGACAACCACTACC
TGAGCACCCAGTCCGCCCTG
AGCAAAGACCCCAACGAGAAGCGCGATCACATGGTCCTGCTGGAGTTCGT
GACCGCCGCCGGGATCACTC
TCGGCATGGACGAGCTGTACAAGCAGTTCAATGAGGATGGCTCTTTCATC
GGCCAATACGGAGGCGCGCC
AATGACCAGCAAGGTGTACGACCCCGAGCAGAGGAAGAGGATGATCACCG
GCCCCCAGTGGTGGGCCAGG
TGCAAGCAGATGAACGTGCTGGACAGCTTCATCAACTACTACGACAGCGA
GAAGCACGCCGAGAACGCCG
TGATCTTCCTGCACGGCAACGCCGCTAGCAGCTACCTGTGGAGGCACGTG
GTGCCCCACATCGAGCCCGT
GGCCAGGTGCATCATCCCCGATCTGATCGGCATGGGCAAGAGCGGCAAGA
GCGGCAACGGCAGCTACAGG
CTGCTGGACCACTACAAGTACCTGACCGCCTGGTTCGAGCTCCTGAACCT
GCCCAAGAAGATCATCTTCG
TGGGCCACGACTGGGGCGCCTGCCTGGCCTTCCACTACAGCTACGAGCAC
CAGGACAAGATCAAGGCCAT
CGTGCACGCCGAGAGCGTGGTGGACGTGATCGAGAGCTGGGACGAGTGGC
CAGACATCGAGGAGGACATC
GCCCTGATCAAGAGCGAGGAGGGCGAGAAGATGGTGCTGGAGAACAACTT
CTTCGTGGAGACCATGCTGC
CCAGCAAGATCATGAGAAAGCTGGAGCCCGAGGAGTTCGCCGCCTACCTG
GAGCCCTTCAAGGAGAAGGG
CGAGGTGAGAAGACCCACCCTGAGCTGGCCCAGAGAGATCCCCCTGGTGA
AGGGCGGCAAGCCCGACGTG
GTGCAGATCGTGAGAAACTACAACGCCTACCTGAGAGCCAGCGACGACCT
GCCCAAGATGTTCATCGAGA
GCGACCCCGGCTTCTTCAGCAACGCCATCGTGGAGGGCGCCAAGAAGTTC
CCCAACACCGAGTTCGTGAA
GGTGAAGGGCCTGCACTTCAGCCAGGAGGACGCCCCCGACGAGATGGGCA
AGTACATCAAGAGCTTCGTG
GAGAGAGTGCTGAAGAACGAGCAGTAAGAATTCGATCTAGACTCGAGTCT
AGAGGGCCCGTTTAAACCCG
CTGATCAGCCTCGACTGTGCCTTCTAGTTGCCAGCCATCTGTTGTTTGCC
CCTCCCCCGTGCCTTCCTTG
ACCCTGGAAGGTGCCACTCCCACTGTCCTTTCCTAATAAAATGAGGAAAT
TGCATCGCATTGTCTGAGTA
GGTGTCATTCTATTCTGGGGGGTGGGGTGGGGCAGGACAGCAAGGGGGAG
GATTGGGAAGACAATAGCAG
GCATGCTGGGGATGCGGTGGGCTCTATGGCTTCTGAGGCGGAAAGAACCA
GCTGGGGCTCTAGGGGGTAT
CCCCACGCGCCCTGTAGCGGCGCATTAAGCGCGGCGGGTGTGGTGGTTAC
GCGCAGCGTGACCGCTACAC
TTGCCAGCGCCCTAGCGCCCGCTCCTTTCGCTTTCTTCCCTTCCTTTCTC
GCCACGTTCGCCGGCTTTCC
CCGTCAAGCTCTAAATCGGGGGCTCCCTTTAGGGTTCCGATTTAGTGCTT
TACGGCACCTCGACCCCAAA
AAACTTGATTAGGGTGATGGTTCACGTAGTGGGCCATCGCCCTGATAGAC
GGTTTTTCGCCCTTTGACGT
TGGAGTCCACGTTCTTTAATAGTGGACTCTTGTTCCAAACTGGAACAACA
CTCAACCCTATCTCGGTCTA
TTCTTTTGATTTATAAGGGATTTTGCCGATTTCGGCCTATTGGTTAAAAA
ATGAGCTGATTTAACAAAAA
TTTAACGCGAATTAATTCTGTGGAATGTGTGTCAGTTAGGGTGTGGAAAG
TCCCCAGGCTCCCCAGCAGG
CAGAAGTATGCAAAGCATGCATCTCAATTAGTCAGCAACCAGGTGTGGAA
AGTCCCCAGGCTCCCCAGCA
GGCAGAAGTATGCAAAGCATGCATCTCAATTAGTCAGCAACCATAGTCCC
GCCCCTAACTCCGCCCATCC
CGCCCCTAACTCCGCCCAGTTCCGCCCATTCTCCGCCCCATGGCTGACTA
ATTTTTTTTATTTATGCAGA
GGCCGAGGCCGCCTCTGCCTCTGAGCTATTCCAGAAGTAGTGAGGAGGCT
TTTTTGGAGGCCTAGGCTTT
TGCAAAAAGCTCCCGGGAGCTTGTATATCCATTTTCGGATCTGATCAGCA
CGTGATGAAAAAGCCTGAAC
TCACCGCGACGTCTGTCGAGAAGTTTCTGATCGAAAAGTTCGACAGCGTC
TCCGACCTGATGCAGCTCTC
GGAGGGCGAAGAATCTCGTGCTTTCAGCTTCGATGTAGGAGGGCGTGGAT
ATGTCCTGCGGGTAAATAGC
TGCGCCGATGGTTTCTACAAAGATCGTTATGTTTATCGGCACTTTGCATC
GGCCGCGCTCCCGATTCCGG
AAGTGCTTGACATTGGGGAATTCAGCGAGAGCCTGACCTATTGCATCTCC
CGCCGTGCACAGGGTGTCAC
GTTGCAAGACCTGCCTGAAACCGAACTGCCCGCTGTTCTGCAGCCGGTCG
CGGAGGCCATGGATGCGATC
GCTGCGGCCGATCTTAGCCAGACGAGCGGGTTCGGCCCATTCGGACCGCA
AGGAATCGGTCAATACACTA
CATGGCGTGATTTCATATGCGCGATTGCTGATCCCCATGTGTATCACTGG
CAAACTGTGATGGACGACAC
CGTCAGTGCGTCCGTCGCGCAGGCTCTCGATGAGCTGATGCTTTGGGCCG
AGGACTGCCCCGAAGTCCGG
CACCTCGTGCACGCGGATTTCGGCTCCAACAATGTCCTGACGGACAATGG
CCGCATAACAGCGGTCATTG
ACTGGAGCGAGGCGATGTTCGGGGATTCCCAATACGAGGTCGCCAACATC
TTCTTCTGGAGGCCGTGGTT
GGCTTGTATGGAGCAGCAGACGCGCTACTTCGAGCGGAGGCATCCGGAGC
TTGCAGGATCGCCGCGGCTC
CGGGCGTATATGCTCCGCATTGGTCTTGACCAACTCTATCAGAGCTTGGT
TGACGGCAATTTCGATGATG
CAGCTTGGGCGCAGGGTCGATGCGACGCAATCGTCCGATCCGGAGCCGGG
ACTGTCGGGCGTACACAAAT
CGCCCGCAGAAGCGCGGCCGTCTGGACCGATGGCTGTGTAGAAGTACTCG
CCGATAGTGGAAACCGACGC
CCCAGCACTCGTCCGAGGGCAAAGGAATAGCACGTGCTACGAGATTTCGA
TTCCACCGCCGCCTTCTATG
AAAGGTTGGGCTTCGGAATCGTTTTCCGGGACGCCGGCTGGATGATCCTC
CAGCGCGGGGATCTCATGCT
GGAGTTCTTCGCCCACCCCAACTTGTTTATTGCAGCTTATAATGGTTACA
AATAAAGCAATAGCATCACA
AATTTCACAAATAAAGCATTTTTTTCACTGCATTCTAGTTGTGGTTTGTC
CAAACTCATCAATGTATCTT
ATCATGTCTGTATACCGTCGACCTCTAGCTAGAGCTTGGCGTAATCATGG
TCATAGCTGTTTCCTGTGTG
AAATTGTTATCCGCTCACAATTCCACACAACATACGAGCCGGAAGCATAA
AGTGTAAAGCCTGGGGTGCC
TAATGAGTGAGCTAACTCACATTAATTGCGTTGCGCTCACTGCCCGCTTT
CCAGTCGGGAAACCTGTCGT
GCCAGCTGCATTAATGAATCGGCCAACGCGCGGGGAGAGGCGGTTTGCGT
ATTGGGCGCTCTTCCGCTTC
CTCGCTCACTGACTCGCTGCGCTCGGTCGTTCGGCTGCGGCGAGCGGTAT
CAGCTCACTCAAAGGCGGTA
ATACGGTTATCCACAGAATCAGGGGATAACGCAGGAAAGAACATGTGAGC
AAAAGGCCAGCAAAAGGCCA
GGAACCGTAAAAAGGCCGCGTTGCTGGCGTTTTTCCATAGGCTCCGCCCC
CCTGACGAGCATCACAAAAA
TCGACGCTCAAGTCAGAGGTGGCGAAACCCGACAGGACTATAAAGATACC
AGGCGTTTCCCCCTGGAAGC
TCCCTCGTGCGCTCTCCTGTTCCGACCCTGCCGCTTACCGGATACCTGTC
CGCCTTTCTCCCTTCGGGAA
GCGTGGCGCTTTCTCATAGCTCACGCTGTAGGTATCTCAGTTCGGTGTAG
GTCGTTCGCTCCAAGCTGGG
CTGTGTGCACGAACCCCCCGTTCAGCCCGACCGCTGCGCCTTATCCGGTA
ACTATCGTCTTGAGTCCAAC
CCGGTAAGACACGACTTATCGCCACTGGCAGCAGCCACTGGTAACAGGAT
TAGCAGAGCGAGGTATGTAG
GCGGTGCTACAGAGTTCTTGAAGTGGTGGCCTAACTACGGCTACACTAGA
AGAACAGTATTTGGTATCTG
CGCTCTGCTGAAGCCAGTTACCTTCGGAAAAAGAGTTGGTAGCTCTTGAT
CCGGCAAACAAACCACCGCT
GGTAGCGGTTTTTTTGTTTGCAAGCAGCAGATTACGCGCAGAAAAAAAGG
ATCTCAAGAAGATCCTTTGA
TCTTTTCTACGGGGTCTGACGCTCAGTGGAACGAAAACTCACGTTAAGGG
ATTTTGGTCATGAGATTATC
AAAAAGGATCTTCACCTAGATCCTTTTAAATTAAAAATGAAGTTTTAAAT
CAATCTAAAGTATATATGAG
TAAACTTGGTCTGACAGTTACCAATGCTTAATCAGTGAGGCACCTATCTC
AGCGATCTGTCTATTTCGTT
CATCCATAGTTGCCTGACTCCCCGTCGTGTAGATAACTACGATACGGGAG
GGCTTACCATCTGGCCCCAG
TGCTGCAATGATACCGCGAGACCCACGCTCACCGGCTCCAGATTTATCAG
CAATAAACCAGCCAGCCGGA
AGGGCCGAGCGCAGAAGTGGTCCTGCAACTTTATCCGCCTCCATCCAGTC
TATTAATTGTTGCCGGGAAG
CTAGAGTAAGTAGTTCGCCAGTTAATAGTTTGCGCAACGTTGTTGCCATT
GCTACAGGCATCGTGGTGTC
ACGCTCGTCGTTTGGTATGGCTTCATTCAGCTCCGGTTCCCAACGATCAA
GGCGAGTTACATGATCCCCC
ATGTTGTGCAAAAAAGCGGTTAGCTCCTTCGGTCCTCCGATCGTTGTCAG
AAGTAAGTTGGCCGCAGTGT
TATCACTCATGGTTATGGCAGCACTGCATAATTCTCTTACTGTCATGCCA
TCCGTAAGATGCTTTTCTGT
GACTGGTGAGTACTCAACCAAGTCATTCTGAGAATAGTGTATGCGGCGAC
CGAGTTGCTCTTGCCCGGCG
TCAATACGGGATAATACCGCGCCACATAGCAGAACTTTAAAAGTGCTCAT
CATTGGAAAACGTTCTTCGG
GGCGAAAACTCTCAAGGATCTTACCGCTGTTGAGATCCAGTTCGATGTAA
CCCACTCGTGCACCCAACTG
ATCTTCAGCATCTTTTACTTTCACCAGCGTTTCTGGGTGAGCAAAAACAG
GAAGGCAAAATGCCGCAAAA
AAGGGAATAAGGGCGACACGGAAATGTTGAATACTCATACTCTTCCTTTT
TCAATATTATTGAAGCATTT
ATCAGGGTTATTGTCTCATGAGCGGATACATATTTGAATGTATTTAGAAA
AATAAACAAATAGGGGTTCC
GCGCACATTTCCCCGAAAAGTGCCACCTGACGTC
SEQ ID NO: 19 (pcFRETchim1)
GACGGATCGGGAGATCTCCCGATCCCCTATGGTGCACTCTCAGTACAATC
TGCTCTGATGCCGCATAGTT
AAGCCAGTATCTGCTCCCTGCTTGTGTGTTGGAGGTCGCTGAGTAGTGCG
CGAGCAAAATTTAAGCTACA
ACAAGGCAAGGCTTGACCGACAATTGCATGAAGAATCTGCTTAGGGTTAG
GCGTTTTGCGCTGCTTCGCG
ATGTACGGGCCAGATATACGCGTTGACATTGATTATTGACTAGTTATTAA
TAGTAATCAATTACGGGGTC
ATTAGTTCATAGCCCATATATGGAGTTCCGCGTTACATAACTTACGGTAA
ATGGCCCGCCTGGCTGACCG
CCCAACGACCCCCGCCCATTGACGTCAATAATGACGTATGTTCCCATAGT
AACGCCAATAGGGACTTTCC
ATTGACGTCAATGGGTGGAGTATTTACGGTAAACTGCCCACTTGGCAGTA
CATCAAGTGTATCATATGCC
AAGTACGCCCCCTATTGACGTCAATGACGGTAAATGGCCCGCCTGGCATT
ATGCCCAGTACATGACCTTA
TGGGACTTTCCTACTTGGCAGTACATCTACGTATTAGTCATCGCTATTAC
CATGGTGATGCGGTTTTGGC
AGTACATCAATGGGCGTGGATAGCGGTTTGACTCACGGGGATTTCCAAGT
CTCCACCCCATTGACGTCAA
TGGGAGTTTGTTTTGGCACCAAAATCAACGGGACTTTCCAAAATGTCGTA
ACAACTCCGCCCCATTGACG
CAAATGGGCGGTAGGCGTGTACGGTGGGAGGTCTATATAAGCAGAGCTCT
CTGGCTAACTAGAGAACCCA
CTGCTTACTGGCTTATCGAAATTAATACGACTCACTATAGGGAGACCCAA
GCTGGCTAGCGTTTAAACTT
AAGCTTGGTACCGAGCTCGGATCCACTAGTCCAGTGTGGTGGAATTCTGC
AGATATCCAGCACAGTGGCG
GCCGCATGGTGAGCAAGGGCGAGGAGCTGTTCACCGGGGTGGTGCCCATC
CTGGTCGAGCTGGACGGCGA
CGTAAACGGCCACAAGTTCAGCGTGTCCGGCGAGGGCGAGGGCGATGCCA
CCTACGGCAAGCTGACCCTG
AAGCTGATCTGCACCACCGGCAAGCTGCCCGTGCCCTGGCCCACCCTCGT
GACCACCCTGGGCTACGGCC
TGCAGTGCTTCGCCCGCTACCCCGACCACATGAAGCAGCACGACTTCTTC
AAGTCCGCCATGCCCGAAGG
CTACGTCCAGGAGCGCACCATCTTCTTCAAGGACGACGGCAACTACAAGA
CCCGCGCCGAGGTGAAGTTC
GAGGGCGACACCCTGGTGAACCGCATCGAGCTGAAGGGCATCGACTTCAA
GGAGGACGGCAACATCCTGG
GGCACAAGCTGGAGTACAACTACAACAGCCACAACGTCTATATCACCGCC
GACAAGCAGAAGAACGGCAT
CAAGGCCAACTTCAAGATCCGCCACAACATCGAGGACGGCGGCGTGCAGC
TCGCCGACCACTACCAGCAG
AACACCCCCATCGGCGACGGCCCCGTGCTGCTGCCCGACAACCACTACCT
GAGCTACCAGTCCGCCCTGA
GCAAAGACCCCAACGAGAAGCGCGATCACATGGTCCTGCTGGAGTTCGTG
ACCGCCGCCGGGATCACTCT
CGGCATGGACGAGCTGTACAAGTCCGGAGGCGCGCCAATGGTGAGCAAGG
GCGAGGAGCTGTTCACCGGG
GTGGTGCCCATCCTGGTCGAGCTGGACGGCGACGTAAACGGCCACAAGTT
CAGCGTGTCCGGCGAGGGCG
AGGGCGATGCCACCTACGGCAAGCTGACCCTGAAGTTCATCTGCACCACC
GGCAAGCTGCCCGTGCCCTG
GCCCACCCTCGTGACCACCCTGACCTGGGGCGTGCAGTGCTTCAGCCGCT
ACCCCGACCACATGAAGCAG
CACGACTTCTTCAAGTCCGCCATGCCCGAAGGCTACGTCCAGGAGCGCAC
CATCTTCTTCAAGGACGACG
GCAACTACAAGACCCGCGCCGAGGTGAAGTTCGAGGGCGACACCCTGGTG
AACCGCATCGAGCTGAAGGG
CATCGACTTCAAGGAGGACGGCAACATCCTGGGGCACAAGCTGGAGTACA
ACTACATCAGCCACAACGTC
TATATCACCGCCGACAAGCAGAAGAACGGCATCAAGGCCAACTTCAAGAT
CCGCCACAACATCGAGGACG
GCAGCGTGCAGCTCGCCGACCACTACCAGCAGAACACCCCCATCGGCGAC
GGCCCCGTGCTGCTGCCCGA
CAACCACTACCTGAGCACCCAGTCCGCCCTGAGCAAAGACCCCAACGAGA
AGCGCGATCACATGGTCCTG
CTGGAGTTCGTGACCGCCGCCGGGATCACTCTCGGCATGGATGAGCTGTA
TAAGTAACTCGAGTCTAGAG
GGCCCGTTTAAACCCGCTGATCAGCCTCGACTGTGCCTTCTAGTTGCCAG
CCATCTGTTGTTTGCCCCTC
CCCCGTGCCTTCCTTGACCCTGGAAGGTGCCACTCCCACTGTCCTTTCCT
AATAAAATGAGGAAATTGCA
TCGCATTGTCTGAGTAGGTGTCATTCTATTCTGGGGGGTGGGGTGGGGCA
GGACAGCAAGGGGGAGGATT
GGGAAGACAATAGCAGGCATGCTGGGGATGCGGTGGGCTCTATGGCTTCT
GAGGCGGAAAGAACCAGCTG
GGGCTCTAGGGGGTATCCCCACGCGCCCTGTAGCGGCGCATTAAGCGCGG
CGGGTGTGGTGGTTACGCGC
AGCGTGACCGCTACACTTGCCAGCGCCCTAGCGCCCGCTCCTTTCGCTTT
CTTCCCTTCCTTTCTCGCCA
CGTTCGCCGGCTTTCCCCGTCAAGCTCTAAATCGGGGGCTCCCTTTAGGG
TTCCGATTTAGTGCTTTACG
GCACCTCGACCCCAAAAAACTTGATTAGGGTGATGGTTCACGTAGTGGGC
CATCGCCCTGATAGACGGTT
TTTCGCCCTTTGACGTTGGAGTCCACGTTCTTTAATAGTGGACTCTTGTT
CCAAACTGGAACAACACTCA
ACCCTATCTCGGTCTATTCTTTTGATTTATAAGGGATTTTGCCGATTTCG
GCCTATTGGTTAAAAAATGA
GCTGATTTAACAAAAATTTAACGCGAATTAATTCTGTGGAATGTGTGTCA
GTTAGGGTGTGGAAAGTCCC
CAGGCTCCCCAGCAGGCAGAAGTATGCAAAGCATGCATCTCAATTAGTCA
GCAACCAGGTGTGGAAAGTC
CCCAGGCTCCCCAGCAGGCAGAAGTATGCAAAGCATGCATCTCAATTAGT
CAGCAACCATAGTCCCGCCC
CTAACTCCGCCCATCCCGCCCCTAACTCCGCCCAGTTCCGCCCATTCTCC
GCCCCATGGCTGACTAATTT
TTTTTATTTATGCAGAGGCCGAGGCCGCCTCTGCCTCTGAGCTATTCCAG
AAGTAGTGAGGAGGCTTTTT
TGGAGGCCTAGGCTTTTGCAAAAAGCTCCCGGGAGCTTGTATATCCATTT
TCGGATCTGATCAGCACGTG
ATGAAAAAGCCTGAACTCACCGCGACGTCTGTCGAGAAGTTTCTGATCGA
AAAGTTCGACAGCGTCTCCG
ACCTGATGCAGCTCTCGGAGGGCGAAGAATCTCGTGCTTTCAGCTTCGAT
GTAGGAGGGCGTGGATATGT
CCTGCGGGTAAATAGCTGCGCCGATGGTTTCTACAAAGATCGTTATGTTT
ATCGGCACTTTGCATCGGCC
GCGCTCCCGATTCCGGAAGTGCTTGACATTGGGGAATTCAGCGAGAGCCT
GACCTATTGCATCTCCCGCC
GTGCACAGGGTGTCACGTTGCAAGACCTGCCTGAAACCGAACTGCCCGCT
GTTCTGCAGCCGGTCGCGGA
GGCCATGGATGCGATCGCTGCGGCCGATCTTAGCCAGACGAGCGGGTTCG
GCCCATTCGGACCGCAAGGA
ATCGGTCAATACACTACATGGCGTGATTTCATATGCGCGATTGCTGATCC
CCATGTGTATCACTGGCAAA
CTGTGATGGACGACACCGTCAGTGCGTCCGTCGCGCAGGCTCTCGATGAG
CTGATGCTTTGGGCCGAGGA
CTGCCCCGAAGTCCGGCACCTCGTGCACGCGGATTTCGGCTCCAACAATG
TCCTGACGGACAATGGCCGC
ATAACAGCGGTCATTGACTGGAGCGAGGCGATGTTCGGGGATTCCCAATA
CGAGGTCGCCAACATCTTCT
TCTGGAGGCCGTGGTTGGCTTGTATGGAGCAGCAGACGCGCTACTTCGAG
CGGAGGCATCCGGAGCTTGC
AGGATCGCCGCGGCTCCGGGCGTATATGCTCCGCATTGGTCTTGACCAAC
TCTATCAGAGCTTGGTTGAC
GGCAATTTCGATGATGCAGCTTGGGCGCAGGGTCGATGCGACGCAATCGT
CCGATCCGGAGCCGGGACTG
TCGGGCGTACACAAATCGCCCGCAGAAGCGCGGCCGTCTGGACCGATGGC
TGTGTAGAAGTACTCGCCGA
TAGTGGAAACCGACGCCCCAGCACTCGTCCGAGGGCAAAGGAATAGCACG
TGCTACGAGATTTCGATTCC
ACCGCCGCCTTCTATGAAAGGTTGGGCTTCGGAATCGTTTTCCGGGACGC
CGGCTGGATGATCCTCCAGC
GCGGGGATCTCATGCTGGAGTTCTTCGCCCACCCCAACTTGTTTATTGCA
GCTTATAATGGTTACAAATA
AAGCAATAGCATCACAAATTTCACAAATAAAGCATTTTTTTCACTGCATT
CTAGTTGTGGTTTGTCCAAA
CTCATCAATGTATCTTATCATGTCTGTATACCGTCGACCTCTAGCTAGAG
CTTGGCGTAATCATGGTCAT
AGCTGTTTCCTGTGTGAAATTGTTATCCGCTCACAATTCCACACAACATA
CGAGCCGGAAGCATAAAGTG
TAAAGCCTGGGGTGCCTAATGAGTGAGCTAACTCACATTAATTGCGTTGC
GCTCACTGCCCGCTTTCCAG
TCGGGAAACCTGTCGTGCCAGCTGCATTAATGAATCGGCCAACGCGCGGG
GAGAGGCGGTTTGCGTATTG
GGCGCTCTTCCGCTTCCTCGCTCACTGACTCGCTGCGCTCGGTCGTTCGG
CTGCGGCGAGCGGTATCAGC
TCACTCAAAGGCGGTAATACGGTTATCCACAGAATCAGGGGATAACGCAG
GAAAGAACATGTGAGCAAAA
GGCCAGCAAAAGGCCAGGAACCGTAAAAAGGCCGCGTTGCTGGCGTTTTT
CCATAGGCTCCGCCCCCCTG
ACGAGCATCACAAAAATCGACGCTCAAGTCAGAGGTGGCGAAACCCGACA
GGACTATAAAGATACCAGGC
GTTTCCCCCTGGAAGCTCCCTCGTGCGCTCTCCTGTTCCGACCCTGCCGC
TTACCGGATACCTGTCCGCC
TTTCTCCCTTCGGGAAGCGTGGCGCTTTCTCATAGCTCACGCTGTAGGTA
TCTCAGTTCGGTGTAGGTCG
TTCGCTCCAAGCTGGGCTGTGTGCACGAACCCCCCGTTCAGCCCGACCGC
TGCGCCTTATCCGGTAACTA
TCGTCTTGAGTCCAACCCGGTAAGACACGACTTATCGCCACTGGCAGCAG
CCACTGGTAACAGGATTAGC
AGAGCGAGGTATGTAGGCGGTGCTACAGAGTTCTTGAAGTGGTGGCCTAA
CTACGGCTACACTAGAAGAA
CAGTATTTGGTATCTGCGCTCTGCTGAAGCCAGTTACCTTCGGAAAAAGA
GTTGGTAGCTCTTGATCCGG
CAAACAAACCACCGCTGGTAGCGGTTTTTTTGTTTGCAAGCAGCAGATTA
CGCGCAGAAAAAAAGGATCT
CAAGAAGATCCTTTGATCTTTTCTACGGGGTCTGACGCTCAGTGGAACGA
AAACTCACGTTAAGGGATTT
TGGTCATGAGATTATCAAAAAGGATCTTCACCTAGATCCTTTTAAATTAA
AAATGAAGTTTTAAATCAAT
CTAAAGTATATATGAGTAAACTTGGTCTGACAGTTACCAATGCTTAATCA
GTGAGGCACCTATCTCAGCG
ATCTGTCTATTTCGTTCATCCATAGTTGCCTGACTCCCCGTCGTGTAGAT
AACTACGATACGGGAGGGCT
TACCATCTGGCCCCAGTGCTGCAATGATACCGCGAGACCCACGCTCACCG
GCTCCAGATTTATCAGCAAT
AAACCAGCCAGCCGGAAGGGCCGAGCGCAGAAGTGGTCCTGCAACTTTAT
CCGCCTCCATCCAGTCTATT
AATTGTTGCCGGGAAGCTAGAGTAAGTAGTTCGCCAGTTAATAGTTTGCG
CAACGTTGTTGCCATTGCTA
CAGGCATCGTGGTGTCACGCTCGTCGTTTGGTATGGCTTCATTCAGCTCC
GGTTCCCAACGATCAAGGCG
AGTTACATGATCCCCCATGTTGTGCAAAAAAGCGGTTAGCTCCTTCGGTC
CTCCGATCGTTGTCAGAAGT
AAGTTGGCCGCAGTGTTATCACTCATGGTTATGGCAGCACTGCATAATTC
TCTTACTGTCATGCCATCCG
TAAGATGCTTTTCTGTGACTGGTGAGTACTCAACCAAGTCATTCTGAGAA
TAGTGTATGCGGCGACCGAG
TTGCTCTTGCCCGGCGTCAATACGGGATAATACCGCGCCACATAGCAGAA
CTTTAAAAGTGCTCATCATT
GGAAAACGTTCTTCGGGGCGAAAACTCTCAAGGATCTTACCGCTGTTGAG
ATCCAGTTCGATGTAACCCA
CTCGTGCACCCAACTGATCTTCAGCATCTTTTACTTTCACCAGCGTTTCT
GGGTGAGCAAAAACAGGAAG
GCAAAATGCCGCAAAAAAGGGAATAAGGGCGACACGGAAATGTTGAATAC
TCATACTCTTCCTTTTTCAA
TATTATTGAAGCATTTATCAGGGTTATTGTCTCATGAGCGGATACATATT
TGAATGTATTTAGAAAAATA
AACAAATAGGGGTTCCGCGCACATTTCCCCGAAAAGTGCCACCTGACGTC
SEQ ID NO: 20 (Venus coding region)
GGATCCACCATGGTGAGCAAGGGCGAGGAGCTGTTCACCGGGGTGGTGCC
CATCCTGGTCGAGCTGGACG
GCGACGTAAACGGCCACAAGTTCAGCGTGTCCGGCGAGGGCGAGGGCGAT
GCCACCTACGGCAAGCTGAC
CCTGAAGCTGATCTGCACCACCGGCAAGCTGCCCGTGCCCTGGCCCACCC
TCGTGACCACCCTGGGCTAC
GGCCTGCAGTGCTTCGCCCGCTACCCCGACCACATGAAGCAGCACGACTT
CTTCAAGTCCGCCATGCCCG
AAGGCTACGTCCAGGAGCGCACCATCTTCTTCAAGGACGACGGCAACTAC
AAGACCCGCGCCGAGGTGAA
GTTCGAGGGCGACACCCTGGTGAACCGCATCGAGCTGAAGGGCATCGACT
TCAAGGAGGACGGCAACATC
CTGGGGCACAAGCTGGAGTACAACTACAACAGCCACAACGTCTATATCAC
CGCCGACAAGCAGAAGAACG
GCATCAAGGCCAACTTCAAGATCCGCCACAACATCGAGGACGGCGGCGTG
CAGCTCGCCGACCACTACCA
GCAGAACACCCCCATCGGCGACGGCCCCGTGCTGCTGCCCGACAACCACT
ACCTGAGCTACCAGTCCGCC
CTGAGCAAAGACCCCAACGAGAAGCGCGATCACATGGTCCTGCTGGAGTT
CGTGACCGCCGCCGGGATCA
CTCTCGGCATGGACGAGCTGTACAAGTAAGAATTC

What is claimed is:

1. A donor-insert-acceptor resonance energy transfer system comprising:
(i) a donor molecule;
(ii) an insert molecule attached to said donor molecule, said insert molecule being a fragment of an actual or putative substrate of an enzyme that post-translationally modifies said substrate or a variant of said substrate; and
(iii) an acceptor molecule attached to said insert molecule;
wherein said insert molecule maintains a predetermined spacing, r, between said donor molecule and said acceptor molecule within the range of 0.7R₀ to 1.1R₀, and wherein said donor molecule emits energy in the presence of a donor activator and said acceptor receives energy from said donor, resulting in a ratio of detectable signal of acceptor divided by detectable signal of said donor, and wherein the co-occurrence of an event modifying said insert molecule to alter said spacing, r, causes a measurable change in the ratio of detectable signals.

2. The donor-insert-acceptor system of claim 1, wherein the donor energy is in the form of fluorescence and the acceptor detectable signal is in the form of fluorescence.

3. (canceled)

4. The donor-insert-acceptor system of claim 1, wherein the insert is between 10 and 27 amino acid residues in length.

5. The donor-insert-acceptor system of claim 1, wherein the variant has up to 12 amino acid residues replaced.

6. The donor-insert-acceptor system of claim 1, wherein the donor and acceptor are polypeptides.

7. The donor-insert-acceptor system of claim 6, wherein the acceptor polypeptide is an autofluorescent polypeptide and the donor protein is a luciferase.

8. The donor-insert-acceptor system of claim 7, wherein the autofluorescent polypeptide is green fluorescent protein 2, the luciferase is Renilla luciferase, and the donor activator molecule is coelenterazine.

9. The donor-insert-acceptor system of claim 8, wherein the insert is a polypeptide comprising the amino acid sequence of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

10. The donor-insert-acceptor system of claim 1, wherein the insert selected from the group consisting of a MAPK pathway recognition site and a Src pathway recognition site.

11. (canceled)

12. A donor-insert-acceptor resonance energy transfer system comprising:
a nucleic acid molecule encoding
(i) a donor molecule;
(ii) an insert molecule attached to said donor molecule, said insert molecule being a fragment of an actual or putative substrate of a tyrosine kinase or a serine/threonine kinase or a variant of said substrate; and
(iii) an acceptor molecule attached to said insert molecule;
wherein said insert molecule maintains a predetermined spacing, r, between said donor molecule and said acceptor molecule within the range of 0.7R₀ to 1.1R₀, and wherein said donor molecule emits energy in the presence of a donor activator and said acceptor molecule displays a detectable signal in response to the emission of energy by said donor molecule and upon the co-occurrence of an event modifying the insert molecule to alter said spacing, r, resulting in a measurable change in said detectable signal, wherein said change is indicative of said event.

13. The donor-insert-acceptor system of claim 12, wherein the donor energy is in the form of fluorescence and the acceptor detectable signal is in the form of fluorescence.

14. (canceled)

15. The donor-insert-acceptor system of claim 12, wherein the nucleic acid molecule is a plasmid.

16. The donor-insert-acceptor system of claim 12, wherein the insert is between 12 and 27 amino acid residues in length.

17. The donor-insert-acceptor system of claim 12, wherein the variant has up to 12 amino acid residues replaced.

18. The donor-insert-acceptor system of claim 12, wherein the acceptor polypeptide is an autofluorescent polypeptide and the donor polypeptide is a luciferase.

19. The donor-insert-acceptor system of claim 18, wherein the autofluorescent polypeptide is green fluorescent protein 2, the luciferase is Renilla luciferase, and the donor activator molecule is coelenterazine.

20. The donor-insert-acceptor system of claim 19, wherein the insert is a polypeptide comprising the amino acid sequence of SEQ ID NO: 1, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11.

21. The donor-insert-acceptor system of claim 12, wherein the insert is selected from the group consisting of a MAPK pathway recognition site and a Src pathway recognition site.

22. (canceled)

23. A method of detecting an event associated with an insert in a donor-insert-acceptor resonance energy transfer system comprising the steps:
(i) measuring a first ratio of detectable signal of a first donor-insert-acceptor system according to claim 1 within a first test sample and a second ratio of detectable signal of a second donor-insert-acceptor system according to claim 1 within a second control sample; and
(ii) determining a signal ratio for the first and second samples;
wherein a difference in the signal ratios indicates that an event associated with the insert has occurred.

24-34. (canceled)

35. A donor-insert-acceptor resonance energy transfer kit comprising:
(i) a first component comprising a donor-insert-acceptor resonance energy transfer system of claim 1; and
(ii) a second component comprising a donor-acceptor resonance energy transfer system which comprises
(a) a second donor molecule; and
(b) a second acceptor molecule attached to the second donor molecule;
wherein the second donor molecule emits energy in the presence of a second donor activator and the second acceptor molecule displays a detectable signal in response to the emission of energy by the donor molecule.


SEQUENCE LISTING (20 sequences)

SEQ ID NO: 1 - protein, 12 aa, Homo sapiens
QFNEDGSFIGQY

SEQ ID NO: 2 - DNA, 7281 bp, artificial
  plasmid
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggaggcgcgccaatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccgctagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggcgcctgcctggccttccactacagctacgagcaccaggacaagatcaaggccatcgtgcacgccgagagcgtggtggacgtgatcgagagctgggacgagtggccagacatcgaggaggacatcgccctgatcaagagcgaggagggcgagaagatggtgctggagaacaacttcttcgtggagaccatgctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagatgttcatcgagagcgaccccggcttcttcagcaacgccatcgtggagggcgccaagaagttccccaacaccgagttcgtgaaggtgaagggcctgcacttcagccaggaggacgcccccgacgagatgggcaagtacatcaagagcttcgtggagagagtgctgaagaacgagcagtaagaattcgatctagactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - protein, 12 aa, artificial
  synthetic peptide
QFNEDGSFIGQD

SEQ ID NO: 4 - protein, 12 aa, artificial
  synthetic peptide
QFNEDGSFIGQH

SEQ ID NO: 5 - protein, 12 aa, artificial
  synthetic peptide
QFNEDGSFIGQF

SEQ ID NO: 6 - protein, 12 aa, artificial
  synthetic peptide
KPLGSDDSLADY

SEQ ID NO: 7 - protein, 25 aa, Homo sapiens
DDSLADYGGSVDVQFNEDGSFIGQY

SEQ ID NO: 8 - protein, 25 aa, artificial
  synthetic peptide
DDSLADYGGSVDVQFNEDGSFIGQH

SEQ ID NO: 9 - protein, 25 aa, artificial
  synthetic peptide
DDSLADYGGSVDVQFNEDGSFIGQF

SEQ ID NO: 10 - protein, 12 aa, Homo sapiens
NEDGSFIGQYSG

SEQ ID NO: 11 - protein, 12 aa, Homo sapiens
DGSFIGQYSGKK

SEQ ID NO: 12 - protein, 5 aa, Homo sapiens
FIGQY

SEQ ID NO: 13 - protein, 12 aa, Homo sapiens
LCFIKRSKGGKY

SEQ ID NO: 14 - protein, 19 aa, artificial
  synthetic peptide
XDPDHDHTGFLTEYVATRW

SEQ ID NO: 15 - protein, 555 aa, artificial
  fusion protein
MVSKGEELFTGVVPILVELDGDVNGHKFSVSGEGEGDATYGKLTLKFICTTGKLPVPWPTLVTTLSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGAPMTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 16 - protein, 493 aa, artificial
  fusion protein
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYKQFNEDGSFIGQYGAPMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 17 - protein, 483 aa, artificial
  fusion protein
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYKSGGAPMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 18 - DNA, 7314 bp, artificial
  recombinant vector (pcBRETchim1.FIGQY)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcagttcaatgaggatggctctttcatcggccaatacggaggcgcgccaatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccgctagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggcgcctgcctggccttccactacagctacgagcaccaggacaagatcaaggccatcgtgcacgccgagagcgtggtggacgtgatcgagagctgggacgagtggccagacatcgaggaggacatcgccctgatcaagagcgaggagggcgagaagatggtgctggagaacaacttcttcgtggagaccatgctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagatgttcatcgagagcgaccccggcttcttcagcaacgccatcgtggagggcgccaagaagttccccaacaccgagttcgtgaaggtgaagggcctgcacttcagccaggaggacgcccccgacgagatgggcaagtacatcaagagcttcgtggagagagtgctgaagaacgagcagtaagaattcgatctagactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 19 - DNA, 7050 bp, artificial
  recombinant vector (pcFRETchim1)
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggaggcgcgccaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggatgagctgtataagtaactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 20 - DNA, 735 bp, artificial
  Venus coding region
ggatccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaagaattc